(19) United States
(12) Patent Application Publication
(10) Pub. No.: US 2015/0005277 A1
(43) Pub. Date: Jan. 1, 2015

(54) Protein Kinase Inhibitors and Uses Thereof

(71) Applicant: Beigene, Ltd., Grand Cayman (KY)

(72) Inventors: Zhiwei Wang, San Diego, CA (US); Yunhang Guo, Beijing (CN)

(73) Assignee: Beigene, Ltd., Grand Cayman (KY)

(21) Appl. No.: 14/311,284

(22) Filed: Jun. 21, 2014

(30) Foreign Application Priority Data
Jun. 28, 2013 (CN) PCT/CN2013/078297

(51) Int. Cl. C07D 487/04; A61K 31/454; C07D 401/14; A61K 31/4545; A61K 31/4045; A61K 31/4155; A61K 31/437; A61K 31/407; C07D 209/42; C07D 401/04; C07D 403/04

(52) U.S. Cl. CPC C07D 487/04; C07D 401/04; A61K 31/454; C07D 401/14; A61K 31/4545; C07D 403/04; A61K 31/4155; A61K 31/437; A61K 31/407; C07D 209/42; A61K 31/4045
USPC 514/210.18; 546/211; 514/326; 546/194; 514/318; 548/357.5; 546/113; 514/300; 548/453; 514/412; 548/483; 514/419; 546/208

(57) ABSTRACT

The invention is compounds of formula (I), and salts thereof, compositions thereof, and methods of use therefor. In particular, disclosed herein are certain compounds that can be useful for inhibiting protein kinase, including Bruton's tyrosine kinase (Btk), and for treating disorders mediated thereby.

### INTRODUCTION

Bruton's tyrosine kinase (Btk) is primarily expressed in most hematopoietic cells such as B cells, mast cells and macrophages (Smith et al., J. Immunol. 152: 557-565, 1994) and is localized in bone marrow, spleen and lymph node tissue. It belongs to the Tec tyrosine kinase family (Vetrie et al., Nature 361: 226-233, 1993; Bradshaw, Cell Signal. 22: 1175-84, 2010). Btk is an essential component of B-cell receptor (BCR) and FcR signaling pathways, which involve in B-cell development, differentiation (Khan, Immunol. Res. 23: 147, 2001). Btk is activated by upstream Src-family kinases. Once activated, Btk in turn phosphorylates PLC gamma, leading to effects on B-cell function and survival (Humphries et al., J. Biol. Chem. 279: 37651, 2004). These signaling pathways must be precisely regulated. Mutations in the gene encoding Btk cause an inherited B-cell specific immunodeficiency disease in humans, known as X-linked agammaglobulinemia (XLA) (Conley et al., Annu. Rev. Immunol. 27: 199-227, 2009). Aberrant BCR-mediated signaling may result in dysregulated B-cell activation leading to a number of autoimmune and inflammatory diseases. Preclinical studies show that Btk deficient mice are resistant to developing collagen-induced arthritis. Moreover, clinical studies of Rituxan, a CD20 antibody to deplete mature B-cells, reveal the key role of B-cells in a number of inflammatory diseases such as rheumatoid arthritis, systemic lupus erythematosus and multiple sclerosis (Gurcan et al., Int. Immunopharmacol. 9: 10-25, 2009). Therefore, Btk inhibitors can be used to treat autoimmune and/or inflammatory diseases.

In addition, it is reported that Btk mediates interactions with the tumor microenvironment and promotes the survival and proliferation of chronic lymphocytic leukemia (CLL) cells (Byrd et al., N. Engl. J. Med. Jun. 19, 2013.) and aberrant activating of Btk plays important role in pathogenesis of B-cell lymphomas (Davis et al., Nature 463: 88-92, 2010). This has indicated that inhibition of Btk is useful in the treatment of hematological malignancies. Preliminary clinical trial results showed that the Bruton's tyrosine kinase (Btk) inhibitor PCI-32765 was effective in treatment of several types of B-cell lymphoma (for example, 54^{th} American Society of Hematology (ASH) annual meeting abstract, December 2012: 686 The Bruton's Tyrosine Kinase (Btk) Inhibitor, Ibrutinib (PCI-32765), Has Preferential Activity in the ABC Subtype of Relapsed/Refractory De Novo Diffuse Large B-Cell Lymphoma (DLBCL): Interim Results of a Multicenter, Open-Label, Phase2 Study; Byrd et al., N. Engl. J. Med. Jun. 19, 2013.). Because Btk plays a central role as a mediator in multiple signal transduction pathways, inhibitors of Btk are of great interest as anti-inflammatory and/or anti-cancer agents (Mohamed et al., Immunol. Rev. 228: 58-73, 2009; Pan, Drug News perspect 21: 357-362, 2008; Rokosz et al., Expert Opin. Ther. Targets 12: 883-903, 2008; Uckun et al., Anti-cancer Agents Med. Chem. 7: 624-632, 2007; Lou et al., J. Med. Chem. 55(10): 4539-4550, 2012).

Small molecule inhibitors of Btk are being developed for anti-inflammatory and anticancer therapy. Ibrutinib (PCI-32765, See: U.S. Pat. No. 7,514,444B2 and related documents, for examples, US2012053189A1; WO 2011153514; WO 2011046964; US2010254905A1; WO2010009342; WO2008121742; WO2008054827; US20080139582; US20080076921; U.S. Pat. No. 7,718,662B1; WO2007087068; US20100035841) is a first-in class of Btk inhibitor, currently undergoing multiple clinical trials in relapsed or refractory mantle cell lymphoma (MCL) and chronic lymphocytic leukaemia (CLL). Another Btk inhibitor entered clinical trials is AVL-292 (See, for example, US 20100249092; US20100029610; US2010016296; US20120077832; WO 2011090760; WO 2010028236; WO 2009158571; WO2009051822; WO2010123870). Ono pharmaceuticals and Mannkind Corporation have been doing clinical trials with their small molecular Btk inhibitors, respectively (See, for example, ONO-4059, WO2011152351; WO2007136790A2).

Other Btk inhibitors are also known. See, for example, US2012/0232054 (LOCUS PHARMACEUTICALS, INC.), WO2010126960 (LOCUS PHARMACEUTICALS, INC.), WO 2011/162515 (HANMI HOLDINGS CO. LTD), WO2012135801 (UNIVERSITY OF UTAH RESEARCH FOUNDATION), Kim et al., Bioorg. Med. Chem. Lett. 21: 6258-6263, 2011 (Pfizer), US8084620B2 (BMS), WO2002050071; WO2008116064; WO2010011837; WO 2011159857 (BMS), US2012058996A1; US2012082702A1; US20100160303 (BMS), US2012129852A1 (BMS), WO 2011019780 (BMS), WO2011029043; WO2011029046 (Biogen Idec), U.S. Pat. No. 7,393,848 (CGI), US20060178367; US20060183746 (CGI), EP2068849 (CGI), WO 2005005429; WO 2005014599; WO 2005047290; WO 2006053121; WO2008033834; WO 2008033858; WO 2006099075; WO 2008033854; WO 2008033857; WO 2009039397 (CGI), WO 2009137596; WO 2010056875; WO 2010068788; WO 2010068806; WO 2010068810 (CGI, GENENTECH), WO 2011140488; WO 2012030990; WO 2012031004 (GILEAD & GENENTECH), US2012040961A1 (DANA-FARBER CANCER INSTITUTE), WO 2005011597; WO 2008045627; WO 2008144253 (IRM LLC), WO 2007140222; WO 2013008095 (NOVARTIS), WO 2012170976A2 (Merck), WO2012135944A1 (PHARMASCIENCE), US2010144705A1; US20120028981A1 (PRINCIPIA BIOPHARMA), WO 2010065898A2; WO 2012158795A1; WO 2012158764A1; WO 2012158810A1 (PRINCIPIA BIOPHARMA), US20090318448A1; US20100016301; US2009105209A1; US20100222325; US20100004231 (ROCHE), WO 2012156334A1; WO 2012020008; WO 2010122038; WO 2010006970; WO 2010006947; WO 2010000633; WO 2009077334; WO 2009098144 (ROCHE), WO 2006065946; WO 2007027594; WO 2007027729 (VERTEX).

### SUMMARY OF THE INVENTION

The invention provides methods and compositions for inhibiting Btk and treating disease associated with undesirable Btk activity (Btk-related diseases).

In one embodiment the invention provides Btk inhibitors or compounds of formula:

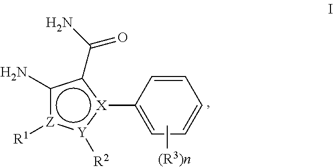

stereoisomers thereof, and pharmaceutically acceptable salts thereof, wherein:

X—Y—Z is N—C—C and R² is present, or C—N—N and R² is absent;

R¹ is 3-8 membered, N-containing ring, wherein the N is unsubstituted or substituted with R⁴;

R² is H or lower alkyl, particularly methyl, ethyl, propyl or butyl; or

R¹ and R², together with the atoms to which they are attached, form a 4-8 membered ring, preferably a 5-6 membered ring, selected from cycloalkyl, saturated or unsaturated heterocycle, aryl, and heteroaryl rings unsubstituted or substituted with at least one substituent L-R⁴;

R³ is in each instance, independently halogen, alkyl, S-alkyl, CN, OR⁵;

n is 1, 2, 3 or 4, preferably 1 or 2;

L is a bond, NH, heteroalkyl, or heterocyclyl;

R⁴ is COR′, CO₂R′, or SO₂R′, wherein R′ is substituted or unsubstituted alkyl, substituted or unsubstituted alkenyl, substituted or unsubstituted alkynyl;

R⁵ is H or unsubstituted or substituted heteroalkyl, alkyl, cycloalkyl, saturated or unsaturated heterocyclyl, aryl, or heteroaryl.

The invention encompasses all combinations of particular embodiments, including:

X—Y—Z is C—N—N and R² is absent; and R¹ is 3-8 membered, N-containing ring, N-substituted with R⁴;

X—Y—Z is N—C—C and R² is present, R¹ is 3-8 membered, N-containing ring, N-substituted with R⁴; and R² is H or lower alkyl;

X—Y—Z is N—C—C and R² is present; and R¹ and R², together with the atoms to which they are attached, form a 4-8 membered ring selected from cycloalkyl, saturated or unsaturated heterocycle, aryl, and heteroaryl rings unsubstituted or substituted with at least one substituent L-R⁴, wherein preferred rings of R¹ and R² are 5-6-membered, particularly dihydropyrrole, tetrahydropyridine, tetrahydroazepine, phenyl, or pyridine;

X—Y—Z is N—C—C and R² is present; and R¹ and R², together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond;

R¹ is piperidine or azaspiro[3.3]heptane, preferably N-substituted with R⁴;

R⁴ is COR′ or SO₂R′, particularly wherein R′ is substituted or unsubstituted alkenyl, particularly substituted or unsubstituted ethenyl; and/or

R⁵ is unsubstituted or substituted alkyl or aryl, particularly substituted or unsubstituted phenyl or methyl, such as cyclopropyl-substituted methyl with or tetrabutyl-substituted phenyl.

The invention provides all combinations of particularly recited embodiments as though each combination had been separately recited, such as the combinations of:

R¹ is piperidine or azaspiro[3.3]heptane, N-substituted with R⁴, wherein R⁴ is H, COR′ or SO₂R′, and R′ is substituted or unsubstituted alkenyl, particularly substituted or unsubstituted ethenyl;

R³ is —OR⁵, R⁵ is phenyl, and n is 1;

R¹ and R², together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond; R³ is —OR⁵; n is 1; R⁴ is COR′, and R′ is ethenyl; and R⁵ is phenyl; and

X—Y—Z is C—N—N and R² is absent; R¹ is piperidine, N-substituted with R⁴; R³ is —OR⁵; n is 1; R⁴ is COR′, and R′ is unsubstituted or substituted alkenyl, particularly ethenyl; and R⁵ is substituted or unsubstituted aryl, particularly phenyl.

The invention provides compounds of the examples herein, and of Table I or II, stereoisomers thereof, and pharmaceutically acceptable salts thereof.

In particular embodiments, the invention provides a compound below, or a pharmaceutically acceptable salt thereof:

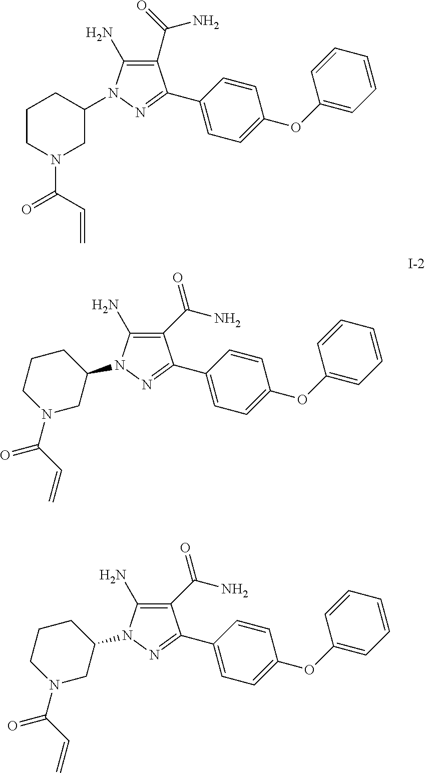 [SMILES: C=CC(=O)N1CCCC(n2nc(-c3ccc(Oc4ccccc4)cc3)c(C(N)=O)c2N)C1 ; C=CC(=O)N1CCC[C@@H](n2nc(-c3ccc(Oc4ccccc4)cc3)c(C(N)=O)c2N)C1 ; C=CC(=O)N1CCC[C@H](n2nc(-c3ccc(Oc4ccccc4)cc3)c(C(N)=O)c2N)C1]

The invention also provides subject compounds having a Btk-inhibiting activity corresponding to an IC50 of 10 uM or less in a Btk Kinase Assay.

The invention also provides pharmaceutical compositions comprising a therapeutically effective amount of a subject compound in unit dosage form and one or more pharmaceutically acceptable carriers.

The invention also provides combinations comprising a therapeutically effective amount of a subject compound and a different agent therapeutically active against an autoimmune, inflammatory disease or cancer.

The invention also provides methods treating a Btk related disease, or disease associated with undesirable Btk activity, particularly an allergic disease, an autoimmune disease (e.g. rheumatoid arthritis), an inflammatory disease, or cancer (e.g. a B-cell proliferative disorder, such as chronic lymphocytic lymphoma, non-Hodgkin's lymphoma, diffuse large B cell lymphoma, mantle cell lymphoma, follicular lymphoma or chronic lymphocytic leukemia), which methods generally comprise administering to a mammal in need thereof an effective amount of a subject compound, an N-oxide thereof or a prodrug thereof, and optionally detecting a resultant amelioration of disease or symptom thereof, or Btk-inhibition.

The invention also provides the subject compounds for use as a medicament, and use of the subject compounds in the manufacture of a medicament for the treatment of a Btk related disease.

### DESCRIPTION OF PARTICULAR EMBODIMENTS OF THE INVENTION

Disclosed herein are compounds that can inhibit tyrosine kinases, such as Btk, Blk, Bmx, EGFR, ERBB2, ERBB4, Itk, Jak3, Tec and Txk kinases.

The following words, phrases and symbols are generally intended to have the meanings as set forth below, except to the extent that the context in which they are used indicates otherwise. The following abbreviations and terms have the indicated meanings throughout.

The term “alkyl” refers to a hydrocarbon group selected from linear and branched saturated hydrocarbon groups of 1-18, or 1-12, or 1-6 carbon atoms. Examples of the alkyl group include methyl, ethyl,1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one CC triple bond and of 2-18, or 2-12, or 2-6 carbon atoms. Examples of the alkynyl group include ethynyl, 1-propynyl, 2-propynyl(propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “Aryl” herein refers to a group selected from:5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap with heteroaryl, separately defined below. Hence, if one or more carbocyclic aromatic rings are fused with a heterocyclic aromatic ring, the resulting ring system is heteroaryl, not aryl, as defined herein.

The term “halogen” or “halo” refers to F, Cl, Br or I.

The term “heteroalkyl” refers to alkyl comprising at least one heteroatom.

The term “heteroaryl” refers to a group selected from:

5- to 7-membered aromatic, monocyclic rings comprising 1, 2, 3 or 4 heteroatoms selected from N, O, and S, with the remaining ring atoms being carbon;

8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyclo[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

Substituents are selected from: halogen, —R′, —OR, ═O, ═NR, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR′—SO₂NR′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, —N₃, —CH(Ph)₂, perfluoro(C1-C4)alkoxy and perfluoro(C1-C4)alkyl, in a number ranging from zero to three, with those groups having zero, one or two substituents being particularly preferred. R′, R″ and R′″ each independently refer to hydrogen, unsubstituted (C1-C8)alkyl and heteroalkyl, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SR′, —SiR′R″R′″, —OC(O)R′, —C(O)R′, —CO₂R′, —CONR′R″, —OC(O)NR′R″, —NR″C(O)R′, —NR″CO₂R′, —NR′—SO₂NR″R′″, —S(O)R′, —SO₂R′, —SO₂NR′R″, —NR″SO₂R, —CN and —NO₂, perfluoro(C1-C4)alkoxy and perfluoro(C1-C4)alkyl, where R′ and R″ are as defined above.

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds contain olefin double bonds, unless specified otherwise, such double bonds are meant to include both E and Z geometric isomers.

Some of the compounds may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, aluminum, lithium, and ammonium.

In addition, if a compound is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating,” “treat,” or “treatment” refers to administering at least one compound and/or at least one stereoisomer thereof, and/or at least one pharmaceutically acceptable salt thereof to a subject in recognized need thereof that has, for example, cancer.

An “effective amount” refers to an amount of at least one compound and/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The term “at least one substituent” includes, for example, from 1 to 4, such as from 1 to 3, further as 1 or 2, substituents. For example, “at least one substituent L-R⁴” herein includes from 1 to 4, such as from 1 to 3, further as 1 or 2, substituents selected from the list of L-R⁴ as described herein.

The subject compounds and stereoisomers thereof, and pharmaceutically acceptable salts thereof may be employed alone or in combination with at least one other therapeutic agent for treatment. In some embodiments, the compounds, stereoisomers thereof, and pharmaceutically acceptable salts thereof can be used in combination with at least one additional therapeutic agent. The at least one additional therapeutic agent can be, for example, selected from anti-hyperproliferative, anti-cancer, and chemotherapeutic agents. The compound and/or one pharmaceutically acceptable salt disclosed herein may be administered with the at least one other therapeutic agent in a single dosage form or as a separate dosage form. When administered as a separate dosage form, the at least one other therapeutic agent may be administered prior to, at the same time as, or following administration of the compound and/or one pharmaceutically acceptable salt disclosed herein.

A “chemotherapeutic agent” is a chemical compound useful in the treatment of cancer, regardless of mechanism of action. Chemotherapeutic agents include compounds used in “targeted therapy” and conventional chemotherapy. Suitable chemotherapeutic agents can be, for example, selected from: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons, such as IFN-a and interleukins, such as IL-2); adoptive immunotherapy agents; hematopoietic growth factors; agents that induce tumor cell differentiation (e.g., all-trans-retinoic acid); gene therapy reagents; antisense therapy reagents and nucleotides; tumor vaccines; and inhibitors of angiogenesis.

Examples of chemotherapeutic agents include Erlotinib (TARCEVA®, Genentech/OSI Pharm.); Bortezomib (VELCADE®, Millennium Pharm.); Fulvestrant (FASLODEX®, AstraZeneca); Sunitinib (SUTENT®, Pfizer); Letrozole (FEMARA®, Novartis); Imatinib mesylate (GLEEVEC®, Novartis); PTK787/ZK 222584 (Novartis); Oxaliplatin (Eloxatin®, Sanofi); 5-FU (5-fluorouracil); Leucovorin; Rapamycin (Sirolimus, RAPAMUNE®, Wyeth); Lapatinib (TYKERB®, GSK572016, Glaxo Smith Kline); Lonafarnib (SCH 66336); Sorafenib (NEXAVAR®, Bayer); Irinotecan (CAMPTOSAR®, Pfizer) and Gefitinib (IRESSA®, AstraZeneca); AG1478, AG1571 (SU 5271, Sugen); alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines such as altretamine, triethylenemelamine, triethylenephosphoramide, triethylenethiophosphoramide and trimethylomelamine; acetogenins (such as bullatacin and bullatacinone); a camptothecin (such as the synthetic analog topotecan); bryostatin; callystatin; CC-1065 and its adozelesin, carzelesin and bizelesin synthetic analogs; cryptophycins (such as cryptophycin 1 and cryptophycin 8); dolastatin; duocarmycin and the synthetic analogs thereof, such as KW-2189 and CB1-TM1; eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, such as calicheamicin gamma1I and calicheamicin omegaI1 (Angew Chem. Intl. Ed. Engl. (1994) 33:183-186); dynemicin, such as dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminol evulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (such as T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

The “chemotherapeutic agent” can also be selected, for example, from: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, such as those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above.

The “chemotherapeutic agent” can also be selected, for example, from therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with a subject compound and stereoisomers thereof, and pharmaceutically acceptable salt thereof may, for example, be selected from: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Also provided is a composition comprising a subject compound and stereoisomers thereof, and pharmaceutically acceptable salts thereof, and at least one pharmaceutically acceptable carrier.

The composition comprising a subject compound and stereoisomers thereof, and pharmaceutically acceptable salts thereof can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The subject compounds and stereoisomers thereof, and pharmaceutically acceptable salts thereof can be administered orally in solid dosage forms, such as capsules, tablets, troches, dragées, granules and powders, or in liquid dosage forms, such as elixirs, syrups, emulsions, dispersions, and suspensions. The subject compounds and stereoisomers thereof, and pharmaceutically acceptable salts thereof disclosed herein can also be administered parenterally, in sterile liquid dosage forms, such as dispersions, suspensions or solutions. Other dosages forms that can also be used to administer the subject compounds and stereoisomers thereof, and pharmaceutically acceptable salts thereof disclosed herein as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules containing the compound and/or the at least one pharmaceutically acceptable salt thereof disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene gycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol, a standard reference text in the art.

The subject compounds and stereoisomers thereof, and pharmaceutically acceptable salts thereof disclosed herein can further be examined for efficacy in treating Btk related diseases by in vivo assays. For example, the compound and/or the at least one pharmaceutically acceptable salts thereof disclosed herein can be administered to an animal (e.g., a mouse model) having Btk related diseases and its therapeutic effects can be accessed. Positive results in one or more of such tests are sufficient to increase the scientific storehouse of knowledge and hence sufficient to demonstrate practical utility of the compounds and/or salts tested. Based on the results, an appropriate dosage range and administration route for animals, such as humans, can also be determined.

For administration by inhalation, the subject compounds and stereoisomers thereof, and pharmaceutically acceptable salts thereof may be conveniently delivered in the form of an aerosol spray presentation from pressurized packs or nebulisers. The subject compounds and stereoisomers thereof, and pharmaceutically acceptable salts thereof may also be delivered as powders, which may be formulated and the powder composition may be inhaled with the aid of an insufflation powder inhaler device. One exemplary delivery system for inhalation can be metered dose inhalation (MDI) aerosol, which may be formulated as a suspension or solution of a subject compound and stereoisomers thereof, and pharmaceutically acceptable salts thereof disclosed herein in at least one suitable propellant, selected, for example, from fluorocarbons and hydrocarbons.

For ocular administration, an ophthalmic preparation may be formulated with an appropriate weight percentage of a solution or suspension of the subject compound and stereoisomers thereof, and pharmaceutically acceptable salts thereof in an appropriate ophthalmic vehicle, such that the subject compound and stereoisomers thereof, and at least one pharmaceutically acceptable salts thereof is maintained in contact with the ocular surface for a sufficient time period to allow the compound to penetrate the corneal and internal regions of the eye.

Useful pharmaceutical dosage-forms for administration of the subject compounds and stereoisomers thereof, and pharmaceutically acceptable salts thereof disclosed herein include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, a large number of unit capsules can be prepared by filling standard two-piece hard gelatin capsules each with, for example, 100 milligrams of the subject compound and stereoisomers thereof, and pharmaceutically acceptable salt thereof disclosed herein in powder, 150 milligrams of lactose, 50 milligrams of cellulose, and 6 milligrams magnesium stearate.

In some embodiments, a mixture of the compound, stereoisomers thereof, and pharmaceutically acceptable salts thereof a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, a large number of tablets can be prepared by conventional procedures so that the dosage unit comprises, for example, 100 milligrams of the compound, stereoisomers thereof, and pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, and pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, stereoisomers thereof, and pharmaceutically acceptable salts thereof are administered stepwise or in conjunction with at least one other therapeutic agent. When drugs are administered in physical combination, the dosage form and administration route should be selected depending on the compatibility of the combined drugs. Thus the term coadministration is understood to include the administration of at least two agents concomitantly or sequentially, or alternatively as a fixed dose combination of the at least two active components.

The compounds, stereoisomers thereof, and pharmaceutically acceptable salt thereof disclosed herein can be administered as the sole active ingredient or in combination with at least one second active ingredient, selected, for example, from other active ingredients known to be useful for treating Btk related diseases in a patient.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

General Reaction Scheme for Compound Preparation

The subject compounds and pharmaceutically acceptable salts thereof, can be prepared from (a) commercially available starting materials (b) known starting materials which may be prepared as described in literature procedures (c) new intermediates described in the schemes and experimental procedures herein. In making the compounds of the invention, the order of synthetic steps may be varied to increase the yield of desired product. Some of compounds in this invention may be generated by the methods as shown in the following reaction schemes and the description thereof.

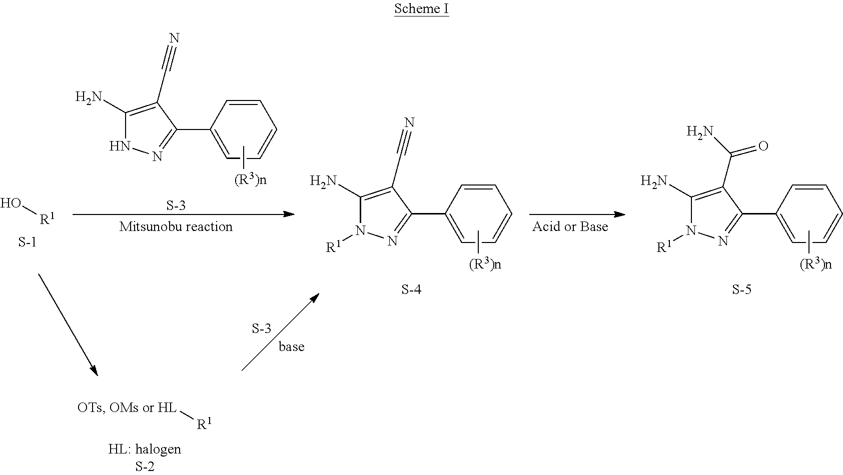

Scheme I above shows a general synthetic route that is used for preparing the compound S-5 of this invention, where R¹, R³ and n are as described herein. Intermediate S-3 may be prepared by methods substantially similar to those described in International Patent Publication No. WO 2001/019829 and No. WO 2011/046964. Mitsunobu reaction of alcohol S-1 and pyrazole S-3 afforded the key intermediate S-4. Alternatively, the key intermediate S-4 could be prepared from nucleophilic substitution of the activated intermediate S-2 by pyrazole S-3 in inert solvent such as DMF, acetonitrile or acetone with presence of base such as Cs₂CO₃, K₂CO₃, DIEA or TEA. The nitrile hydrolysis of key intermediate S-4 under alkaline condition such as NaOH or KOH plus H₂O₂ in alcohol, or under acid condition such as H₃PO₄, H₂SO₄, or BF₃.HOAc, yielded the carboxamide S-5.

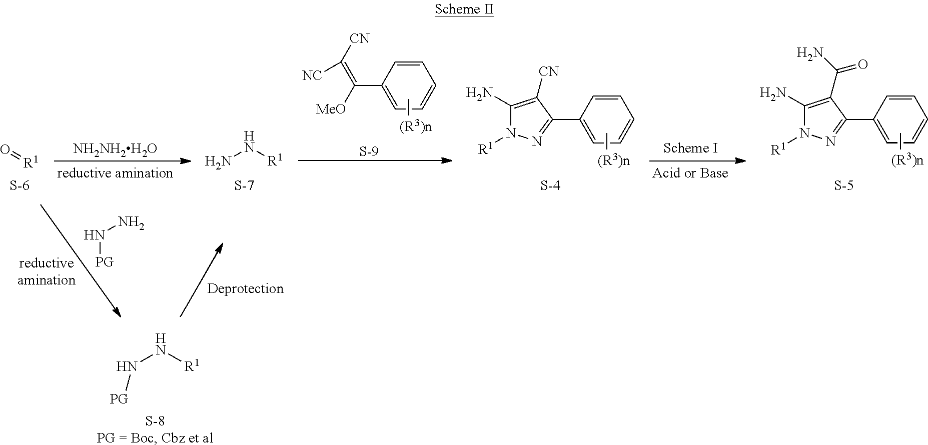

Scheme II above shows an alternative synthetic route to prepare carboxamide S-5 of this invention, where R¹, R³ and n are as described herein. Briefly, reductive amination of ketone S-6 with hydrazine hydrate or protected hydrazine gave intermediate S-7 or S-8. Deprotection of S-8 generated S-7. S-7 was condensed with intermediate S-9 (S-9 also described in International Patent Publication No. WO 2001/019829 and No. WO 2011/046964) in alkaline solvent such as TEA/ethanol to afford pyrazole S-4, which was converted to carboxamide S-5 by the same methods as described in Scheme I.

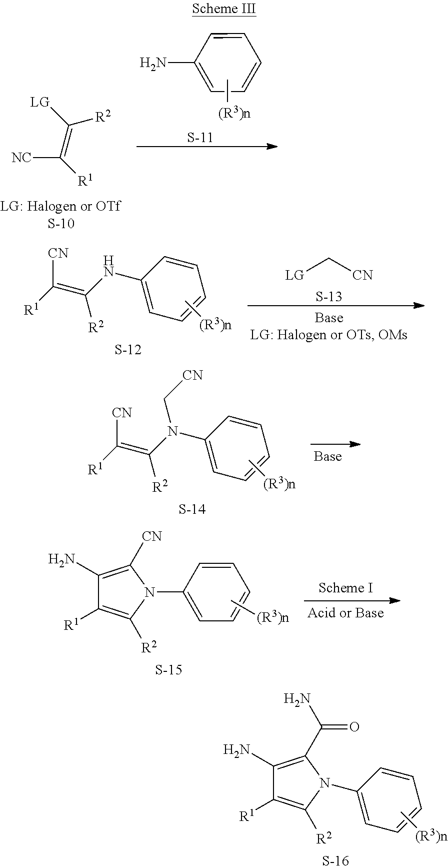

Scheme III above shows a general synthetic route to prepare carboxamide S-16 of this invention, where R¹, R², R³ and n are as described herein. Nucleophilic substitution of S-10 by aniline derivative S-11 gave the corresponding arylamino derivative S-12 in inert solvent such as DMF, acetonitrile or acetone with presence of base such as Cs₂CO₃, K₂CO₃, DIEA or TEA. S-12 further reacted with nitrile S-13 to give the non-isolable dinitrile derivative S-14 also in alkaline solvent as described herein. Intramolecular cyclo-condensation of S-14 and hydrolysis of the nitrile S-15 gave the carboxamide S-16.

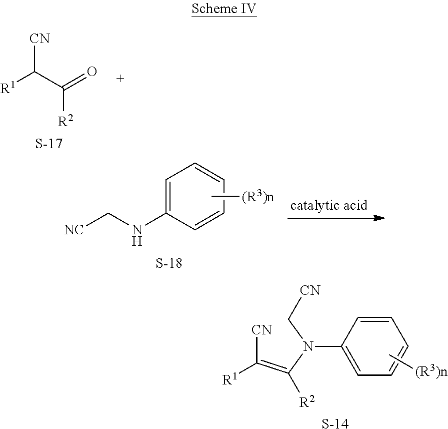

Scheme IV above shows an alternative synthetic route to prepare S-14 of this invention, where R¹, R², R³ and n are as described herein. Arylamino acetonitrile S-18 may be prepared by methods substantially similar to those described in International Patent Publication No. WO 2001/019828. S-18 was condensed with the cyano ketone or aldehyde S-17 to afford dinitrile 5-14 in an inert solvent such as toluene, acetonitrile or dimethoxyethane with the presence of catalytic acid (such as acetic acid or tosylic acid), at 80° C. to reflux temperature for several hours.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise.

Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on a Agilent instrument operating at 400 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d6-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LC-MS spectrometer (Agilent 1260) Detector: MWD (190-400 nm), Mass detector: 6120 SQ

Mobile phase: A: acetonitrile with 0.1% Formic acid, B: water with 0.1% Formic acid

### Column: Poroshell 120 EC-C18, 4.6×50 mm, 2.7 μm

Gradient method: Flow: 1.8 mL/min

| Time | | |
|---|---|---|
| (min) | A (%) | B (%) |
| 0.00 | 5 | 95 |
| 1.5 | 95 | 5 |
| 2.0 | 95 | 5 |
| 2.1 | 5 | 95 |
| 3.0 | 5 | 95 |

Preparative HPLC was conducted on a column (150×21.2 mm ID, 5 μm, Gemini NX-C18) at a flow rate of 20 ml/min, injection volume 2 ml, at room temperature and UV Detection at 214 nm and 254 nm

In the following examples, the abbreviations below are used:
Brine Saturated aqueous sodium chloride solution
DBN Diazabicyclononene
DCM Dichloromethane
DIEA or DIPEA N,N-diisopropylethylamine
DMF N,N-Dimethylformamide
EA Ethyl acetate
HATU O-(7-Azabenzotriazol-1-yl)-N,N,N′,N′-tetramethyluronium hexafluorophosphate
HOAc Acetic acid
H₂O₂ Hydrogen peroxide solution 30% (w/w) in H₂O
Pd/C Palladium on carbon powder
PE Petroleum ether
Pre-TLC Prepared thin layer chromatography
sat. Saturated
THF Tetrahydrofuran
TEA Triethylamine
TsCl p-Methylbenzenesulfonyl chloride
t-BuOH t-Butanol
t-BuONa Sodium tert-butoxide

### Example 1

### Synthesis of Compounds I-1 to I-21

### Compound I-1: 1-(1-Acryloylpiperidin-3-yl)-5-amino-3-(4-phenoxyphenyl)-1H-pyrazole-4-carboxamide

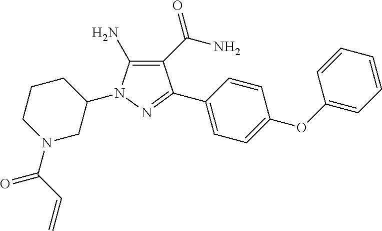 [SMILES: C=CC(=O)N1CCCC(n2nc(-c3ccc(Oc4ccccc4)cc3)c(C(N)=O)c2N)C1]

### Step 1: 2-(Hydroxy(4-phenoxyphenyl)methylene)malononitrile

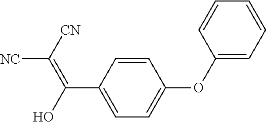 [SMILES: N#CC(C#N)=C(O)c1ccc(Oc2ccccc2)cc1]

A solution of 4-phenoxybenzoic acid (300 g, 1.4 mol) in SOCl₂ (1.2 L) was stirred at 80° C. under N₂ for 3 hr. The mixture was concentrated in vacuum to give the intermediate (315 g) which was used for next step without further purification.

To a solution of propanedinitrile (89.5 g, 1355 mmol) and DIEA (350 g, 2710 mmol) in THF (800 mL) was dropwise a solution of the intermediate (315 g) in toluene (800 mL) at 0-5° C. over 2 hr. The resultant mixture was allowed to warm to RT and stirred for 16 hr. The reaction was quenched with water (2.0 L) and extracted with of EA (2.0 L×3). The combined organic layers were washed with 1000 mL of 3 N HCl aqueous solution, brine (2.0 L×3), dried over Na₂SO₄ and concentrated to give the crude product (330 g, 93%). ¹H NMR (400 MHz, DMSO-d₆) δ 7.62 (d, J=8.8 Hz, 2H), 7.46-7.38 (m, 2H), 7.18 (t, J=7.6 Hz, 1H), 7.06 (d, J=8.0 Hz, 2H), 6.94 (d, J=8.8 Hz, 2H). MS (ESI) m/e [M+1]⁺ 262.9.

### Step 2: 2-(Methoxy(4-phenoxyphenyl)methylene)malononitrile

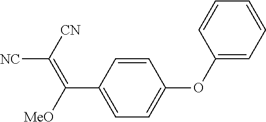 [SMILES: COC(=C(C#N)C#N)c1ccc(Oc2ccccc2)cc1]

A solution of 2-(hydroxy(4-phenoxyphenyl)methylene)malononitrile (50 g, 190.8 mmol) in CH(OMe)₃ (500 mL) was heated to 75° C. for 16 hr. Then the mixture was concentrated to a residue and washed with MeOH (50 mL) to give 25 g (47.5%) of 2-(methoxy(4-phenoxyphenyl)methylene)malononitrile as a yellow solid. ¹H NMR (400 MHz, DMSO-d₆) δ 7.70 (d, J=8.4 Hz, 2H), 7.52-7.45 (m, 2H), 7.28 (t, J=7.6 Hz, 1H), 7.22-7.06 (m, 4H), 3.93 (s, 3H). MS (ESI) m/e [M+1]⁺ 276.9.

### Step 3: 5-Amino-3-(4-phenoxyphenyl)-1H-pyrazole-4-carbonitrile

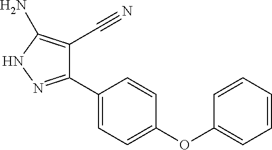 [SMILES: N#Cc1c(-c2ccc(Oc3ccccc3)cc2)n[nH]c1N]

To a solution of 2-(methoxy(4-phenoxyphenyl)methylene)malononitrile (80 g, 290 mmol) in ethanol (200 mL) was added hydrazine hydrate (20 mL). The mixture was stirred at RT for 16 hr then was concentrated to give the crude product and washed with MeOH (30 mL) to afford 55 g (68.8%) of 5-amino-3-(4-phenoxyphenyl)-1H-pyrazole-4-carbonitrile as a off-white solid. ¹H NMR (400 MHz, DMSO-d₆) δ 12.11 (br s, 1H), 7.80 (d, J=8.8 Hz, 2H), 7.46-7.39 (m, 2H), 7.18 (t, J=7.6 Hz, 1H), 7.12-7.04 (m, 4H), 6.43 (br s, 2H).

### Step 4: tert-Butyl 3-(tosyloxy)piperidine-1-carboxylate

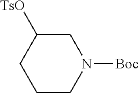 [SMILES: Cc1ccc(S(=O)(=O)OC2CCCN(C(=O)OC(C)(C)C)C2)cc1]

To a solution of tert-butyl 3-hydroxypiperidine-1-carboxylate (1.05 g, 5.0 mmol) in pyridine (8 mL) was added TsCl (1.425 g, 7.5 mmol). The mixture was stirred at RT under N₂ for two days. The mixture was concentrated and partitioned between 100 mL of EA and 100 mL of HCl (1 N) aqueous solution. Organic layer was separated from aqueous layer, washed with saturated NaHCO₃ aqueous solution (100 mL×2), brine (100 mL×3) and dried over Na₂SO₄. Concentrated to afford 1.1 g (60%) of tert-butyl 3-(tosyloxy)piperidine-1-carboxylate as a colorless oil. ¹H NMR (400 MHz, DMSO-d₆) δ 7.80 (d, J=8.0 Hz, 2H), 7.48 (d, J=8.0 Hz, 2H), 4.50-4.40 (m, 1H), 3.70-3.40 (m, 2H), 3.50-2.95 (m, 2H), 2.42 (s, 3H), 1.78-1.66 (m, 1H), 1.65-1.48 (m, 2H), 1.45-1.37 (m, 1H), 1.35 (s, 9H).

### Step 5: tert-Butyl 3-(5-amino-4-cyano-3-(4-phenoxyphenyl)-1H-pyrazol-1-yl)piperidine-1-carboxylate

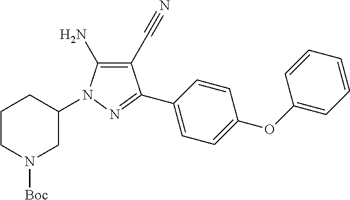 [SMILES: CC(C)(C)OC(=O)N1CCCC(n2nc(-c3ccc(Oc4ccccc4)cc3)c(C#N)c2N)C1]

To a solution of tert-butyl 3-(tosyloxy)piperidine-1-carboxylate (355 mg, 1.0 mmol) and 5-amino-3-(4-phenoxyphenyl)-1H-pyrazole-4-carbonitrile (276 mg, 1.0 mmol) in 5 mL of DMF was added Cs₂CO₃ (650 mg, 2.0 mmol). The mixture was stirred at RT for 16 h, 75° C. for 3 h and 60° C. for 16 h. The mixture was concentrated washed with brine (100 mL×3) and dried over Na₂SO₄. Concentrated and purified by chromatography column on silica gel (eluted with PE/EA=3/1) to afford 60 mg (13%) of tert-butyl 3-(5-amino-4-cyano-3-(4-phenoxyphenyl)-1H-pyrazol-1-yl)piperidine-1-carboxylate as a yellow oil. ¹H NMR (400 MHz, DMSO-d₆) δ 7.78-7.72 (m, 2H), 7.43-7.35 (m, 2H), 7.17-7.11 (m, 1H), 7.08-6.99 (m, 4H), 6.74 (s, 2H), 4.22-4.11 (m, 1H), 4.05-3.70 (m, 2H), 3.35-3.00 (m, 1H), 2.84 (t, J=12.0 Hz, 1H), 2.00-1.72 (m, 3H), 1.55-1.40 (m, 1H), 1.28 (s, 9H). MS (ESI) m/e [M+1]⁺ 460.2.

### Step 6: tert-Butyl 3-(5-amino-4-carbamoyl-3-(4-phenoxyphenyl)-1H-pyrazol-1-yl)piperidine-1-carboxylate

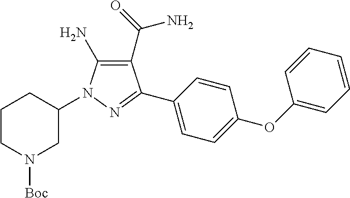 [SMILES: CC(C)(C)OC(=O)N1CCCC(n2nc(-c3ccc(Oc4ccccc4)cc3)c(C(N)=O)c2N)C1]

To a solution of tert-butyl 3-(5-amino-4-cyano-3-(4-phenoxyphenyl)-1H-pyrazol-1-yl)piperidine-1-carboxylate (100 mg, 0.22 mmol) in DMSO (2 mL) and ethanol (2 mL) was added the solution of NaOH (200 mg, 5 mmol) in water (1 mL) and H₂O₂ (1 mL). The mixture was stirred at 60° C. for 15 min Concentrated to remove EtOH, 10 mL of water and 50 mL of EA were added. Organic layer was separated from aqueous layer, washed with brine (30 mL×3) and dried over Na₂SO₄. Concentrated, 50 mg of residue was used directly in the next step, 50 mg of residue was purified by Pre-TLC (eluted with PE/EA=1/1) to afford 12 mg (30%) of tert-butyl 3-(5-amino-4-carbamoyl-3-(4-phenoxyphenyl)-1H-pyrazol-1-yl)piperidine-1-carboxylate as a white solid. ¹H NMR (400 MHz, DMSO-d₆) δ 7.46 (d, J=8.4 Hz, 2H), 7.39 (d, J=8.0 Hz, 1H), 7.37 (d, J=8.0 Hz, 1H), 7.14 (t, J=7.6 Hz, 1H), 7.08-6.98 (m, 4H), 6.37 (s, 2H), 4.21-4.08 (m, 1H), 4.05-3.78 (m, 2H), 3.25-2.95 (m, 1H), 2.75 (t, J=12.0 Hz, 1H), 2.02-1.66 (m, 3H), 1.55-1.40 (m, 1H), 1.36 (s, 9H). MS (ESI) m/e [M+1]⁺ 478.2.

### Step 7: 5-Amino-3-(4-phenoxyphenyl)-1-(piperidin-3-yl)-1H-pyrazole-4-carboxamide

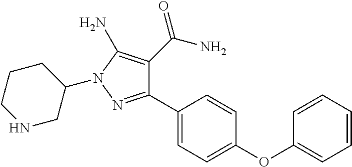 [SMILES: NC(=O)c1c(-c2ccc(Oc3ccccc3)cc2)nn(C2CCCNC2)c1N]

To a solution of tert-butyl 3-(5-amino-4-carbamoyl-3-(4-phenoxyphenyl)-1H-pyrazol-1-yl)piperidine-1-carboxylate (50 mg, 0.11 mmol) in EA (1 mL) was added con. HCl (0.75 mL). The mixture was stirred at RT for 1 h. Then saturated NaHCO₃ was added until PH>7, followed by EA (50 mL). Organic layer was separated from aqueous layer, washed with brine (50 mL×3) and dried over Na₂SO₄. Concentrated and purified by Pre-TLC (eluted with DCM/MeOH/NH₃—H₂O=5/1/0.01) to afford 10 mg (25%) of 5-amino-3-(4-phenoxyphenyl)-1-(piperidin-3-yl)-1H-pyrazole-4-carboxamide as a white solid. ¹H NMR (400 MHz, DMSO-d₆) δ 7.50-7.45 (m, 2H), 7.41-7.35 (m, 2H), 7.17-7.11 (m, 1H), 7.07-6.99 (m, 4H), 6.41 (s, 2H), 4.57-4.44 (m, 1H), 3.39-3.32 (m, 1H), 3.27-3.21 (m, 1H), 3.20-3.13 (m, 1H), 2.96-2.86 (m, 1H), 2.06-1.94 (m, 1H), 1.92-1.70 (m, 3H). MS (ESI) m/e [M+1]⁺ 378.2.

### Step 8: 1-(1-Acryloylpiperidin-3-yl)-5-amino-3-(4-phenoxyphenyl)-1H-pyrazole-4-carboxamide

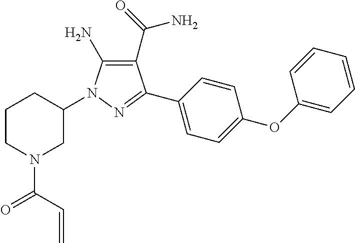 [SMILES: C=CC(=O)N1CCCC(n2nc(-c3ccc(Oc4ccccc4)cc3)c(C(N)=O)c2N)C1]

To a solution of 5-amino-3-(4-phenoxyphenyl)-1-(piperidin-3-yl)-1H-pyrazole-4-carboxamide (63 mg, 0.17 mmol) in DCM (4 mL) was added pyridine (27 mg, 0.34 mmol). Then a solution of acryloyl chloride (12 mg, 0.17 mmol) in DCM (1 mL) was added dropwise. After stirring at RT for 4 h, the mixture was partitioned between 100 mL of DCM and 100 mL of brine. Organic layer was separated from aqueous layer, washed with brine (100 mL×2) and dried over Na₂SO₄. Concentrated and purified by Pre-TLC (eluted with DCM/MeOH=10/1) to afford 4 mg (5.5%) of 1-(1-acryloylpiperidin-3-yl)-5-amino-3-(4-phenoxyphenyl)-1H-pyrazole-4-carboxamide as a white solid. ¹H NMR (400 MHz, CD₃OD-d₄) δ 7.44-7.37 (m, 2H), 7.33-7.26 (m, 2H), 7.09-7.03 (m, 1H), 7.01-6.93 (m, 4H), 6.76-6.60 (m, 1H), 6.17-6.03 (m, 1H), 5.72-5.58 (m, 1H), 4.60-4.51 (m, 0.5H), 4.38-4.29 (m, 0.5H), 4.20-4.00 (m, 2H), 3.58-3.48 (m, 0.5H), 3.11-3.01 (m, 1H), 2.85-2.74 (m, 0.5H), 2.15-1.97 (m, 2H), 1.92-1.83 (m, 1H), 1.63-1.45 (m, 1H). MS (ESI) m/e [M+1]⁺ 432.2.

### Compound I-2: (R)-1-(1-Acryloylpiperidin-3-yl)-5-amino-3-(4-phenoxyphenyl)-1H-pyrazole-4-carboxamide

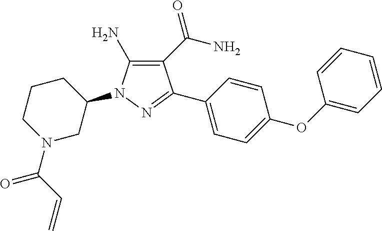 [SMILES: C=CC(=O)N1CCC[C@@H](n2nc(-c3ccc(Oc4ccccc4)cc3)c(C(N)=O)c2N)C1]

The desired product was prepared from 5-amino-3-(4-phenoxyphenyl)-1H-pyrazole-4-carbonitrile and (S)-tert-butyl 3-hydroxypiperidine-1-carboxylate to the similar procedures (step 4 to 8) for compound I-1 under appropriate conditions recognized by one of ordinary skill in the art. ¹H NMR (400 MHz, DMSO-d₆) δ 7.50-7.43 (m, 2H), 7.42-7.34 (m, 2H), 7.17-7.10 (m, 1H), 7.08-6.99 (m, 4H), 6.88-6.71 (m, 1H), 6.37 (s, 2H), 6.17-5.98 (m, 1H), 5.72-5.56 (m, 1H), 4.55-3.98 (m, 3H), 3.50-3.38 (m, 0.5H), 3.07-2.91 (m, 1H), 2.75-2.60 (m, 0.5H), 2.02-1.86 (m, 2H), 1.86-1.76 (m, 1H), 1.54-1.34 (m, 1H). MS (ESI) m/e [M+1]⁺ 432.2.

### Compound I-3: (S)-1-(1-Acryloylpiperidin-3-yl)-5-amino-3-(4-phenoxyphenyl)-1H-pyrazole-4-carboxamide

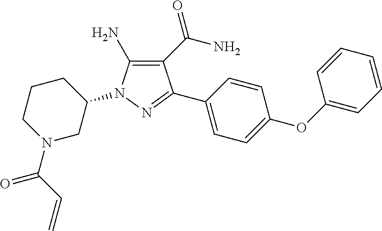 [SMILES: C=CC(=O)N1CCC[C@H](n2nc(-c3ccc(Oc4ccccc4)cc3)c(C(N)=O)c2N)C1]

The desired product was prepared from 5-amino-3-(4-phenoxyphenyl)-1H-pyrazole-4-carbonitrile and (R)-tert-butyl 3-hydroxypiperidine-1-carboxylate according to the similar procedures (step 4 to 8) for compound I-1 under appropriate conditions recognized by one of ordinary skill in the art. MS (ESI) m/e [M+1]⁺ 432.2.

### Compound I-4: (E)-5-Amino-1-(1-(4-(dimethylamino)but-2-enoyl)piperidin-3-yl)-3-(4-phenoxyphenyl)-1H-pyrazole-4-carboxamide

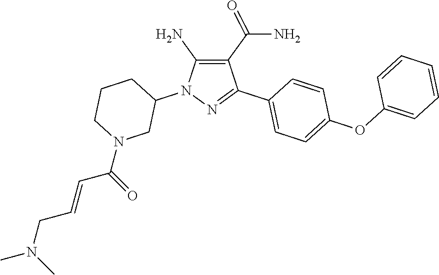 [SMILES: CN(C)C/C=C/C(=O)N1CCCC(n2nc(-c3ccc(Oc4ccccc4)cc3)c(C(N)=O)c2N)C1]

To a solution of 5-amino-3-(4-phenoxyphenyl)-1-(piperidin-3-yl)-1H-pyrazole-4-carboxamide (75 mg, 0.2 mmol) and (E)-4-(dimethylamino)but-2-enoic acid (23 mg, 0.2 mmol) in DMF (2 mL) was added HATU (76 mg, 0.2 mmol) and DIEA (52 mg, 0.4 mmol). After stirring at RT for 16 h, the mixture was concentrated and partitioned between 100 mL of EA and 100 mL of brine. Organic layer was separated from aqueous layer, washed with brine (100 mL×2) and dried over Na₂SO₄. Concentrated and purified by Pre-TLC (eluted with DCM/MeOH=10/1) to afford 25 mg (26%) of (E)-5-amino-1-(1-(4-(dimethylamino)but-2-enoyl)piperidin-3-yl)-3-(4-phenoxyphenyl)-1H-pyrazole-4-carboxamide as a white solid. ¹H NMR (400 MHz, DMSO-d₆) δ 7.49-7.42 (m, 2H), 7.41-7.35 (m, 2H), 7.17-7.10 (m, 1H), 7.07-6.99 (m, 4H), 6.73-6.46 (m, 2H), 6.38 (br s, 2H), 4.50-3.95 (m, 3H), 3.48-3.40 (m, 0.5H), 3.18-3.08 (m, 2H), 3.07-2.94 (m, 1H), 2.76-2.65 (m, 0.5H), 2.21 (s, 6H), 2.00-1.75 (m, 3H), 1.54-1.34 (m, 1H). MS (ESI) m/e [M+1]⁺ 489.2.

### Compound I-5: 5-Amino-1-(1-(but-2-enoyl)piperidin-3-yl)-3-(4-phenoxyphenyl)-1H-pyrazole-4-carboxamide

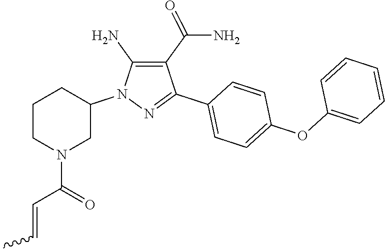

The desired product was prepared from 5-amino-3-(4-phenoxyphenyl)-1-(piperidin-3-yl)-1H-pyrazole-4-carboxamide and but-2-enoyl chloride according to the similar procedure in step 8 of compound I-1 under appropriate conditions recognized by one of ordinary skill in the art. ¹H NMR (400 MHz, DMSO-d₆) δ 7.46 (d, J=8.0 Hz, 2H), 7.41-7.34 (m, 2H), 7.17-7.10 (m, 1H), 7.07-6.99 (m, 4H), 6.72-6.46 (m, 2H), 6.38 (br s, 2H), 4.55-3.95 (m, 3H), 3.48-3.36 (m, 0.5H), 3.05-2.89 (m, 1H), 2.70-2.58 (m, 0.5H), 2.00-1.87 (m, 2H), 1.85-1.72 (m, 4H), 1.50-1.30 (m, 1H). MS (ESI) m/e [M+1]⁺ 446.1.

### Compound I-6: 5-Amino-1-(1-(2-chloroacetyl)piperidin-3-yl)-3-(4-phenoxyphenyl)-1H-pyrazole-4-carboxamide

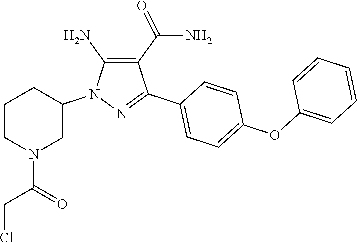 [SMILES: NC(=O)c1c(-c2ccc(Oc3ccccc3)cc2)nn(C2CCCN(C(=O)CCl)C2)c1N]

The desired product was prepared from 5-amino-3-(4-phenoxyphenyl)-1-(piperidin-3-yl)-1H-pyrazole-4-carboxamide and 2-chloroacetyl chloride according to the similar procedure in step 8 of compound I-1 under appropriate conditions recognized by one of ordinary skill in the art. ¹H NMR (400 MHz, DMSO-d₆) δ 7.49-7.43 (m, 2H), 7.42-7.33 (m, 2H), 7.17-7.10 (m, 1H), 7.07-6.99 (m, 4H), 6.38 (s, 2H), 4.51-4.08 (m, 4H), 3.88-3.74 (m, 1H), 3.43-3.33 (m, 0.5H), 3.10-2.90 (m, 1H), 2.73-2.61 (m, 0.5H), 2.02-1.70 (m, 3H), 1.65-1.33 (m, 1H). MS (ESI) m/e [M+1]⁺ 454.1.

### Compound I-7: 5-Amino-1-(1-(1-methyl-1,2,3,6-tetrahydropyridine-4-carbonyl)piperidin-3-yl)-3-(4-phenoxyphenyl)-1H-pyrazole-4-carboxamide

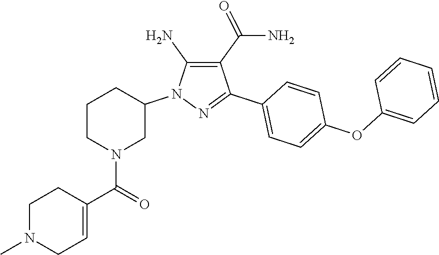 [SMILES: CN1CC=C(C(=O)N2CCCC(n3nc(-c4ccc(Oc5ccccc5)cc4)c(C(N)=O)c3N)C2)CC1]

The desired product was prepared from 5-amino-3-(4-phenoxyphenyl)-1-(piperidin-3-yl)-1H-pyrazole-4-carboxamide and 1-methyl-1,2,3,6-tetrahydropyridine-4-carboxylic acid according to the similar procedure for compound I-4 under appropriate conditions recognized by one of ordinary skill in the art. ¹H NMR (400 MHz, DMSO-d₆) δ 7.59-7.53 (m, 2H), 7.51-7.43 (m, 2H), 7.27-7.20 (m, 1H), 7.17-7.09 (m, 4H), 6.47 (s, 2H), 5.82 (s, 1H), 4.40-3.85 (m, 3H), 3.30-2.82 (m, 4H), 2.66-2.50 (m, 2H), 2.42-2.16 (m, 5H), 2.12-2.00 (m, 2H), 1.97-1.87 (m, 1H), 1.64-1.47 (m, 1H). MS (ESI) m/e [M+1]⁺ 501.2.

### Compound I-8: 5-Amino-3-(4-phenoxyphenyl)-1-(1-(vinylsulfonyl)piperidin-3-yl)-1H-pyrazole-4-carboxamide

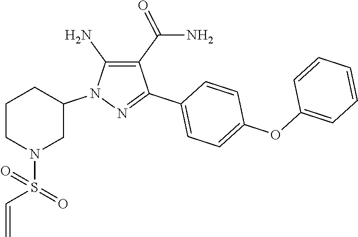 [SMILES: C=CS(=O)(=O)N1CCCC(n2nc(-c3ccc(Oc4ccccc4)cc3)c(C(N)=O)c2N)C1]

To a solution of 2-chloroethanesulfonyl chloride (33 mg, 0.2 mmol) in DCM (2 mL) was added dropwise a solution of 5-amino-3-(4-phenoxyphenyl)-1-(piperidin-3-yl)-1H-pyrazole-4-carboxamide (75 mg, 0.2 mmol) and TEA (40 mg, 0.4 mmol) in DCM (2 mL) at 0° C. After stirring at 0° C. for 2 h and RT for 16 h, the mixture was partitioned between 40 mL of DCM and 100 mL of brine. Organic layer was separated from aqueous layer, washed with brine (100 mL×2) and dried over Na₂SO₄. Concentrated and purified by Pre-TLC (eluted with DCM/MeOH=10/1) to afford 25 mg (27%) of 5-amino-3-(4-phenoxyphenyl)-1-(1-(vinylsulfonyl)piperidin-3-yl)-1H-pyrazole-4-carboxamide as a white solid. ¹H NMR (400 MHz, DMSO-d₆) δ 7.58-7.52 (m, 2H), 7.51-7.44 (m, 2H), 7.23 (t, J=7.4 Hz, 1H), 7.17-7.08 (m, 4H), 6.93 (dd, J=16.8, 10.4 Hz, 1H), 6.52 (s, 2H), 6.22 (d, J=10.4 Hz, 1H), 6.18 (d, J=16.8 Hz, 1H), 4.50-4.38 (m, 1H), 3.72-3.55 (m, 2H), 2.98 (t, J=11.4 Hz, 1H), 2.66 (t, J=11.4 Hz, 1H), 2.10-1.83 (m, 3H), 1.77-1.61 (m, 1H). MS (ESI) m/e [M+1]⁺468.1.

### Compound I-9: 5-Amino-1-(1-(2-cyanoacetyl)piperidin-3-yl)-3-(4-phenoxyphenyl)-1H-pyrazole-4-carboxamide

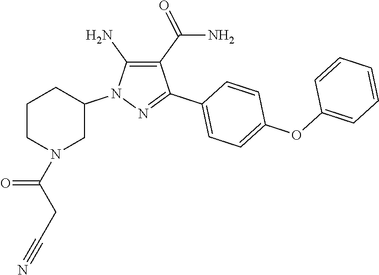 [SMILES: N#CCC(=O)N1CCCC(n2nc(-c3ccc(Oc4ccccc4)cc3)c(C(N)=O)c2N)C1]

The desired product was prepared from 5-amino-3-(4-phenoxyphenyl)-1-(piperidin-3-yl)-1H-pyrazole-4-carboxamide and 2-cyanoacetic acid according to the similar procedure for compound I-4 under appropriate conditions recognized by one of ordinary skill in the art. ¹H NMR (400 MHz, DMSO-d₆) δ 7.53-7.46 (m, 2H), 7.45-7.38 (m, 2H), 7.17 (t, J=7.4 Hz, 1H), 7.11-7.02 (m, 4H), 6.42 (s, 2H), 4.44-4.00 (m, 4H), 3.77-3.61 (m, 1H), 3.42-3.36 (m, 0.5H), 3.11-2.98 (m, 1H), 2.78-2.64 (m, 0.5H), 2.02-1.90 (m, 2H), 1.86-1.73 (m, 1H), 1.67-1.37 (m, 1H). MS (ESI) m/e [M+1]⁺445.2.

### Compound I-10: (E)-5-Amino-1-(1-(4-(dimethylamino)-4-oxobut-2-enyl)piperidin-3-yl)-3-(4-phenoxyphenyl)-1H-pyrazole-4-carboxamide

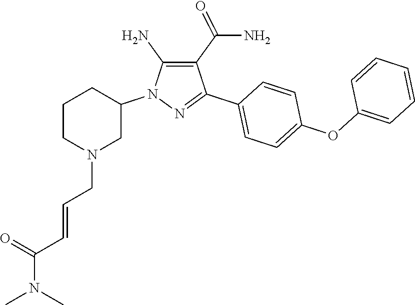 [SMILES: CN(C)C(=O)/C=C/CN1CCCC(n2nc(-c3ccc(Oc4ccccc4)cc3)c(C(N)=O)c2N)C1]

### Step 1: (E)-4-Bromobut-2-enoyl chloride

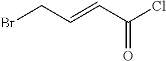

The solution of (E)-4-bromobut-2-enoic acid (165 mg, 1.0 mmol) in thionyl chloride (1 mL) was stirred at 80° C. for 16 h. Concentrated to afford 183 mg (100%) (E)-4-bromobut-2-enoyl chloride as a colorless oil, which was used directly in the next step without further purification.

### Step 2: (E)-4-Bromo-N,N-dimethylbut-2-enamide

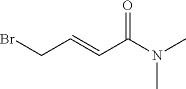

To a solution of dimethylamine in tetrahydrofuran (2 mol/L, 0.5 mL, 1.0 mmol) in DCM (10 mL) was added TEA (202 mg, 2.0 mmol). Then, (E)-4-bromobut-2-enoyl chloride (183 mg, 1.0 mmol) was added dropwise. After stirring at RT for 5 min, brine (20 mL) was added to the above mixture. Organic layer was separated from aqueous layer, washed with brine (20 mL×2) and dried over Na₂SO₄. Concentrated and purified by chromatography column on silica gel (eluted with PE/EA=2/1) to afford 35 mg (18%) of (E)-4-bromo-N,N-dimethylbut-2-enamide as a yellow oil. MS (ESI) m/e [M+1]⁺192.0, 194.0.

### Step 3: (E)-5-Amino-1-(1-(4-(dimethylamino)-4-oxobut-2-enyl)piperidin-3-yl)-3-(4-phenoxyphenyl)-1H-pyrazole-4-carboxamide

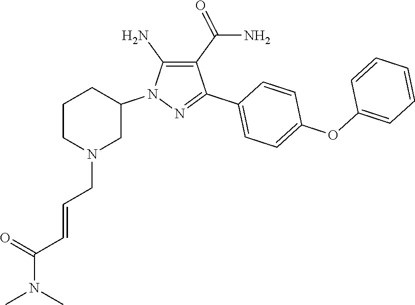 [SMILES: CN(C)C(=O)/C=C/CN1CCCC(n2nc(-c3ccc(Oc4ccccc4)cc3)c(C(N)=O)c2N)C1]

To a solution of 5-amino-3-(4-phenoxyphenyl)-1-(piperidin-3-yl)-1H-pyrazole-4-carboxamide (68 mg, 0.18 mmol) in acetonitrile (10 mL) was added (E)-4-bromo-N,N-dimethylbut-2-enamide (35 mg, 0.18 mmol) and K₂CO₃ (50 mg, 0.36 mmol). After stirring at RT for 16 h, the mixture was partitioned between 50 mL of EA and 50 mL of brine. Organic layer was separated from aqueous layer, washed with brine (50 mL×2) and dried over Na₂SO₄. Concentrated and purified by Pre-TLC (eluted with DCM/MeOH=10/1) to afford 30 mg (34%) of (E)-5-amino-1-(1-(4-(dimethylamino)-4-oxobut-2-enyl)piperidin-3-yl)-3-(4-phenoxyphenyl)-1H-pyrazole-4-carboxamide as a white solid. ¹H NMR (400 MHz, DMSO-d₆) δ 7.44 (d, J=8.4 Hz, 2H), 7.41-7.35 (m, 2H), 7.17-7.10 (m, 1H), 7.07-6.99 (m, 4H), 6.62-6.48 (m, 2H), 6.36 (s, 2H), 4.30-4.18 (m, 1H), 3.19-3.07 (m, 2H), 2.98 (s, 3H), 2.82 (s, 3H), 2.89-2.73 (m, 2H), 2.34-2.33 (m, 1H), 2.00-1.80 (m, 2H), 1.77-1.64 (m, 2H), 1.63-1.49 (m, 1H). MS (ESI) m/e [M+1]⁺ 489.2.

### Compound I-11: (E)-5-Amino-1-(1-(3-cyanoallyl)piperidin-3-yl)-3-(4-phenoxyphenyl)-1H-pyrazole-4-carboxamide and compound I-12: (Z)-5-amino-1-(1-(3-cyanoallyl)piperidin-3-yl)-3-(4-phenoxyphenyl)-1H-pyrazole-4-carboxamide

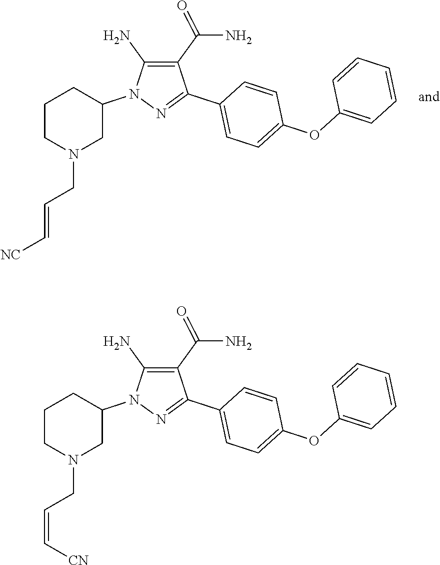 [SMILES: N#C/C=C/CN1CCCC(n2nc(-c3ccc(Oc4ccccc4)cc3)c(C(N)=O)c2N)C1 ; N#C/C=C\CN1CCCC(n2nc(-c3ccc(Oc4ccccc4)cc3)c(C(N)=O)c2N)C1]

Two desired products was prepared from 5-amino-3-(4-phenoxyphenyl)-1-(piperidin-3-yl)-1H-pyrazole-4-carboxamide and 4-bromobut-2-enenitrile according to the similar procedure in step 3 of compound I-10 under appropriate conditions recognized by one of ordinary skill in the art.

Compound I-11: (E)-5-Amino-1-(1-(3-cyanoallyl)piperidin-3-yl)-3-(4-phenoxyphenyl)-1H-pyrazole-4-carboxamide. ¹H NMR (400 MHz, DMSO-d₆) δ 7.47 (d, J=8.4 Hz, 2H), 7.45-7.38 (m, 2H), 7.17 (t, J=7.4 Hz, 1H), 7.10-7.02 (m, 4H), 6.93-6.80 (m, 1H), 6.40 (s, 2H), 5.89 (d, J=16.0 Hz, 1H), 4.32-4.18 (m, 1H), 3.25-3.10 (m, 2H), 2.87 (d, J=10.4 Hz, 1H), 2.78 (d, J=10.4 Hz, 1H), 2.35 (t, J=10.2 Hz, 1H), 1.98 (t, J=10.2 Hz, 1H), 1.92-1.83 (m, 1H), 1.81-1.68 (m, 2H), 1.67-1.54 (m, 1H). MS (ESI) m/e [M+1]⁺ 443.2.

Compound I-12: (Z)-5-Amino-1-(1-(3-cyanoallyl)piperidin-3-yl)-3-(4-phenoxyphenyl)-1H-pyrazole-4-carboxamide. ¹H NMR (400 MHz, DMSO-d₆) δ 7.48 (d, J=8.4 Hz, 2H), 7.45-7.38 (m, 2H), 7.17 (t, J=7.4 Hz, 1H), 7.10-7.02 (m, 4H), 6.78-6.66 (m, 1H), 6.40 (s, 2H), 5.82 (d, J=11.2 Hz, 1H), 4.32-4.18 (m, 1H), 3.34-3.30 (m, 2H), 2.90 (d, J=10.4 Hz, 1H), 2.80 (d, J=10.4 Hz, 1H), 2.39 (t, J=10.2 Hz, 1H), 2.00 (t, J=10.2 Hz, 1H), 2.06-1.94 (m, 1H), 1.93-1.83 (m, 1H), 1.81-1.69 (m, 2H), 1.68-1.54 (m, 1H). MS (ESI) m/e [M+1]⁺ 443.2.

### Compound I-13: 5-Amino-3-(4-phenoxyphenyl)-1-(1-propioloylpiperidin-3-yl)-1H-pyrazole-4-carboxamide

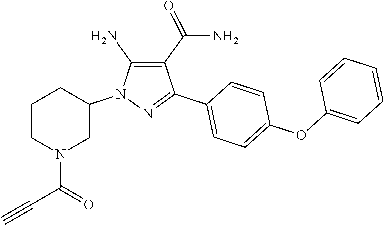 [SMILES: C#CC(=O)N1CCCC(n2nc(-c3ccc(Oc4ccccc4)cc3)c(C(N)=O)c2N)C1]

The desired product was prepared from 5-amino-3-(4-phenoxyphenyl)-1-(piperidin-3-yl)-1H-pyrazole-4-carboxamide and propiolic acid according to the similar procedure for compound I-4 under appropriate conditions recognized by one of ordinary skill in the art. ¹H NMR (400 MHz, DMSO-d₆) δ 7.49-7.44 (m, 2H), 7.41-7.35 (m, 2H), 7.17-7.10 (m, 1H), 7.07-6.99 (m, 4H), 6.39 (br s, 2H), 4.58-4.18 (m, 1H), 4.36-4.11 (m, 3H), 3.0-3.526 (m, 0.5H), 3.23-3.14 (m, 0.5H), 3.13-3.05 (m, 0.5H), 2.85-2.75 (m, 0.5H), 2.01-1.75 (m, 3H), 1.58-1.32 (m, 1H). MS (ESI) m/e [M+1]⁺ 430.1.

### Compound I-14: (R)-1-(1-Acryloylpiperidin-3-yl)-5-amino-3-(4-chlorophenyl)-1H-pyrazole-4-carboxamide

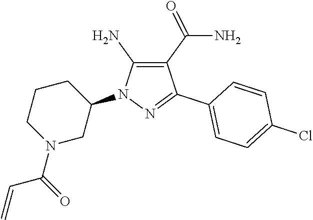 [SMILES: C=CC(=O)N1CCC[C@@H](n2nc(-c3ccc(Cl)cc3)c(C(N)=O)c2N)C1]

The desired product was prepared from 4-chlorobenzoic acid and (S)-tert-butyl 3-hydroxypiperidine-1-carboxylate according to the similar procedure in step 1 to step 8 of compound I-1 under appropriate conditions recognized by one of ordinary skill in the art. ¹H NMR (400 MHz, DMSO-d₆) δ 7.57-7.40 (m, 4H), 6.92-6.70 (m, 1H), 6.35 (s, 2H), 6.19-6.01 (m, 1H), 5.74-5.60 (m, 1H), 4.55-4.46 (m, 0.5H), 4.37-4.29 (m, 0.5H), 4.29-4.01 (m, 2H), 3.52-3.41 (m, 0.5H), 3.14-2.97 (m, 1H), 2.81-2.70 (m, 0.5H), 2.08-1.75 (m, 3H), 1.57-1.39 (m, 1H). MS (ESI) m/e [M+1]⁺ 373.9, 375.9.

### Compound I-15: (R)-1-(1-Acryloylpiperidin-3-yl)-5-amino-3-(3-methoxy-4-methylphenyl)-1H-pyrazole-4-carboxamide

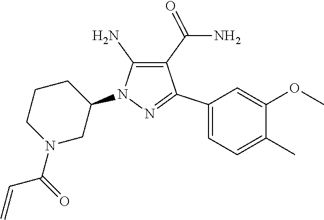 [SMILES: C=CC(=O)N1CCC[C@@H](n2nc(-c3ccc(C)c(OC)c3)c(C(N)=O)c2N)C1]

The desired product was prepared from 3-methoxy-4-methylbenzoic acid and (5)-tert-butyl 3-hydroxypiperidine-1-carboxylate according to the similar procedure for step 1 to step 8 of compound I-1 under appropriate conditions recognized by one of ordinary skill in the art. ¹H NMR (400 MHz, DMSO-d₆) δ 7.21 (d, J=7.6 Hz, 1H), 7.02-6.92 (m, 2H), 6.91-6.73 (m, 1H), 6.45 (s, 2H), 6.21-6.02 (m, 1H), 5.75-5.60 (m, 1H), 4.55-4.45 (m, 0.5H), 4.36-4.28 (m, 0.5H), 4.28-4.00 (m, 2H), 3.80 (s, 3H), 3.55-3.42 (m, 0.5H), 3.15-3.00 (m, 1H), 2.85-2.71 (m, 1H), 2.18 (s, 3H), 2.04-1.78 (m, 3H), 1.55-1.35 (m, 1H). MS (ESI) m/e [M+1]⁺ 383.9.

### Compound I-16: (R)-1-(1-Acryloylpiperidin-3-yl)-5-amino-3-(4-methoxyphenyl)-1H-pyrazole-4-carboxamide

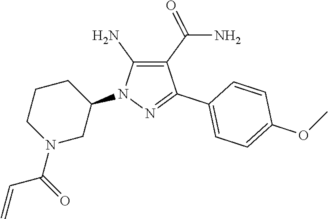 [SMILES: C=CC(=O)N1CCC[C@@H](n2nc(-c3ccc(OC)cc3)c(C(N)=O)c2N)C1]

### Step 1: (R)-tert-Butyl 3-(5-amino-3-(4-(benzyloxy)phenyl)-4-cyano-1H-pyrazol-1-yl)piperidine-1-carboxylate

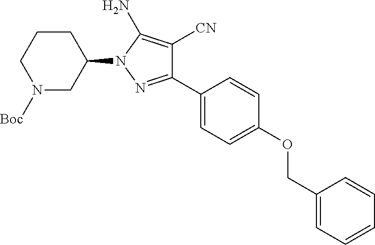 [SMILES: CC(C)(C)OC(=O)N1CCC[C@@H](n2nc(-c3ccc(OCc4ccccc4)cc3)c(C#N)c2N)C1]

The desired product was prepared from 4-(benzyloxy)benzoic acid and according to the similar procedures in step 1 to step 5 of tert-butyl 3-(5-amino-4-cyano-3-(4-phenoxyphenyl)-1H-pyrazol-1-yl)piperidine-1-carboxylate under appropriate conditions recognized by one of ordinary skill in the art. ¹H NMR (400 MHz, DMSO-d₆) δ 7.78 (d, J=8.8 Hz, 2H), 7.60-7.36 (m, 5H), 7.17 (d, J=8.8 Hz, 2H), 6.81 (s, 2H), 5.21 (s, 2H), 4.32-4.18 (m, 1H), 4.00-3.75 (m, 2H), 3.02-2.84 (m, 1H), 2.04-1.87 (m, 3H), 1.65-1.35 (m, 2H), 1.45 (s, 9H).

### Step 2: (R)-tert-Butyl 3-(5-amino-3-(4-(benzyloxy)phenyl)-4-carbamoyl-1H-pyrazol-1-yl)piperidine-1-carboxylate

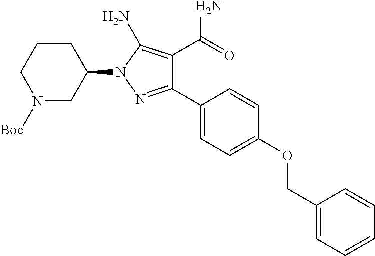 [SMILES: CC(C)(C)OC(=O)N1CCC[C@@H](n2nc(-c3ccc(OCc4ccccc4)cc3)c(C(N)=O)c2N)C1]

To a solution of (R)-tert-butyl 3-(5-amino-3-(4-(benzyloxy)phenyl)-4-cyano-1H-pyrazol-1-yl)piperidine-1-carboxylate (1.2 g, 2.54 mmol) in 30 mL of EtOH was added H₂O₂ (4.0 mL) and DMSO (4.0 mL), then 5N NaOH (5.0 mL) was added dropwise. The resulting mixture was heated to 65° C. for 2 h. The reaction mixture was cooled to RT, diluted with 50 mL of water, then extracted with EtOAc (30 mL×3). The combined organic phase was dried over anhydrous Na₂SO₄, filtered and concentrated in vacuo to give 563 mg (45.1%) of crude product as a gray solid. MS (ESI) m/e [M+1]⁺ 492.0.

### Step 3: (R)-tert-Butyl 3-(5-amino-4-carbamoyl-3-(4-hydroxyphenyl)-1H-pyrazol-1-yl)piperidine-1-carboxylate

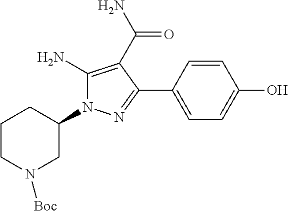 [SMILES: CC(C)(C)OC(=O)N1CCC[C@@H](n2nc(-c3ccc(O)cc3)c(C(N)=O)c2N)C1]

To a solution of (R)-tert-butyl 3-(5-amino-3-(4-(benzyloxy)phenyl)-4-carbamoyl-1H-pyrazol-1-yl)piperidine-1-carboxylate (563 mg, 1.14 mmol) in 20 mL of MeOH was added 100 mg of 10% w/w Pd/C, the resulting mixture was replaced with H₂ for 3 times, then the reaction mixture was stirred at RT under H₂ for 16 h. The reaction mixture was filtered to remove Pd/C, the filtrate was concentrated under reduced pressure to give 384 mg (84%) of desired compound as a gray solid. ¹H NMR (400 MHz, DMSO-d₆) δ 9.68 (br s, 1H), 7.26 (d, J=8.5 Hz, 2H), 6.82 (d, J=8.5 Hz, 2H), 6.42 (s, 2H), 4.20-3.79 (m, 3H), 2.82-2.71 (m, 1H), 2.00-1.71 (m, 3H), 1.55-1.28 (m, 2H), 1.39 (s, 9H). MS (ESI) m/e [M+1]⁺ 402.0.

### Step 4: (R)-tert-Butyl 3-(5-amino-4-carbamoyl-3-(4-methoxyphenyl)-1H-pyrazol-1-yl)piperidine-1-carboxylate

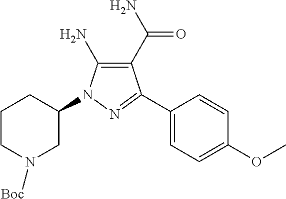 [SMILES: COc1ccc(-c2nn([C@@H]3CCCN(C(=O)OC(C)(C)C)C3)c(N)c2C(N)=O)cc1]

To a solution of (R)-tert-butyl 3-(5-amino-4-carbamoyl-3-(4-hydroxyphenyl)-1H-pyrazol-1-yl)piperidine-1-carboxylate (104 mg, 0.26 mmol) in 30 mL of acetone was added K₂CO₃ (181 mg, 1.3 mmol), then MeI (111 mg, 0.78 mmol) was added, the resulting mixture was stirred at RT for 16 h. The reaction mixture was diluted with 100 mL of water and extracted with EtOAc (50 mL×3). The combined organic phase was dried over anhydrous Na₂SO₄, filtered and concentrated under reduced pressure to give 110 mg (100%) of crude product was obtained as a gray solid. The crude product was used in the next step without further purification. MS (ESI) m/e [M+1]⁺ 416.0.

### Step 5: (R)-1-(1-Acryloylpiperidin-3-yl)-5-amino-3-(4-methoxyphenyl)-1H-pyrazole-4-carboxamide

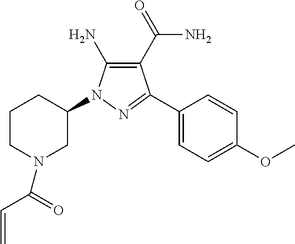 [SMILES: C=CC(=O)N1CCC[C@@H](n2nc(-c3ccc(OC)cc3)c(C(N)=O)c2N)C1]

The desired product was prepared from (R)-tert-butyl 3-(5-amino-4-carbamoyl-3-(4-methoxyphenyl)-1H-pyrazol-1-yl)piperidine-1-carboxylate according to the similar procedures in step 7 to step 8 of compound I-1 under appropriate conditions recognized by one of ordinary skill in the art. ¹H NMR (400 MHz, DMSO-d₆) δ 7.44-7.360 (m, 2H), 7.04-6.98 (m, 2H), 6.92-6.74 (m, 1H), 6.18-6.03 (m, 1H), 5.74-5.60 (m, 1H), 4.55-4.46 (m, 0.5H), 4.38-4.29 (m, 0.5H), 4.27-4.01 (m, 2H), 3.79 (s, 3H), 3.50-3.41 (m, 0.5H), 3.13-2.99 (m, 1H), 2.79-2.68 (m, 0.5H), 2.04-1.93 (m, 2H), 1.89-1.78 (m, 1H), 1.55-1.37 (m, 1H). MS (ESI) m/e [M+1]⁺ 370.0.

### Compound I-17: (R)-1-(1-Acryloylpiperidin-3-yl)-5-amino-3-(4-(cyclopropylmethoxy)phenyl)-1H-pyrazole-4-carboxamide

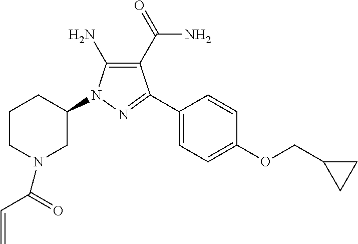 [SMILES: C=CC(=O)N1CCC[C@@H](n2nc(-c3ccc(OCC4CC4)cc3)c(C(N)=O)c2N)C1]

The desired product was prepared from (R)-tert-butyl 3-(5-amino-4-carbamoyl-3-(4-hydroxyphenyl)-1H-pyrazol-1-yl)piperidine-1-carboxylate and (bromomethyl)cyclopropane according to the similar procedures in step 4 to step 5 of compound I-16 under appropriate conditions recognized by one of ordinary skill in the art. ¹H NMR (400 MHz, DMSO-d₆) δ 7.41-7.34 (m, 2H), 7.02-6.96 (m, 2H), 6.90-6.75 (m, 1H), 6.17-6.03 (m, 1H), 5.74-5.60 (m, 1H), 4.54-4.46 (m, 0.5H), 4.38-4.29 (m, 0.5H), 4.27-4.01 (m, 2H), 3.85 (d, J=7.0 Hz, 2H), 3.50-3.41 (m, 0.5H), 3.13-2.99 (m, 1H), 2.79-2.68 (m, 0.5H), 2.04-1.93 (m, 2H), 1.89-1.78 (m, 1H), 1.55-1.37 (m, 1H), 1.28-1.16 (m, 1H), 0.61-0.53 (m, 2H), 0.38-0.29 (m, 2H). MS (ESI) m/e [M+1]⁺ 410.0.

### Compound I-18: (R)-1-(1-Acryloylpiperidin-3-yl)-5-amino-3-(4-isopropoxyphenyl)-1H-pyrazole-4-carboxamide

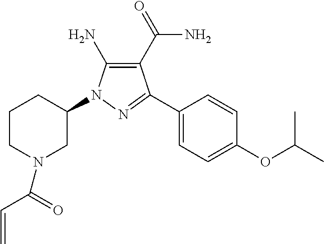 [SMILES: C=CC(=O)N1CCC[C@@H](n2nc(-c3ccc(OC(C)C)cc3)c(C(N)=O)c2N)C1]

The desired product was prepared from (R)-tert-butyl 3-(5-amino-4-carbamoyl-3-(4-hydroxyphenyl)-1H-pyrazol-1-yl)piperidine-1-carboxylate and 2-bromopropane according to the similar procedures in step 4 to step 5 of compound I-16 under appropriate conditions recognized by one of ordinary skill in the art. ¹H NMR (400 MHz, DMSO-d₆) δ 7.41-7.34 (m, 2H), 7.02-6.96 (m, 2H), 6.90-6.73 (m, 1H), 6.41 (s, 2H), 6.20-6.02 (m, 1H), 5.74-5.60 (m, 1H), 4.71-4.60 (m, 1H), 4.55-4.46 (m, 0.5H), 4.39-4.29 (m, 0.5H), 4.27-3.99 (m, 2H), 3.52-3.41 (m, 0.5H), 3.13-2.92 (m, 1H), 2.79-2.65 (m, 0.5H), 2.04-1.93 (m, 2H), 1.89-1.78 (m, 1H), 1.55-1.37 (m, 1H), 1.28 (d, J=6.0 Hz, 6H). MS (ESI) m/e [M+1]⁺ 397.9.

### Compound I-19: 1-(2-Acryloyl-2-azaspiro[3.3]heptan-6-yl)-5-amino-3-(4-phenoxyphenyl)-1H-pyrazole-4-carboxamide

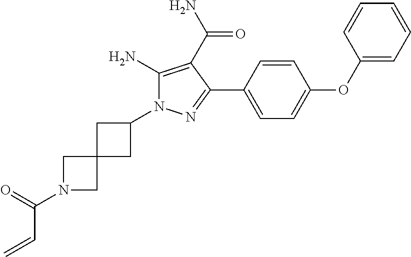 [SMILES: C=CC(=O)N1CC2(CC(n3nc(-c4ccc(Oc5ccccc5)cc4)c(C(N)=O)c3N)C2)C1]

The desired product was prepared from 5-amino-3-(4-phenoxyphenyl)-1H-pyrazole-4-carbonitrile and tert-butyl 6-hydroxy-2-azaspiro[3.3]heptane-2-carboxylate according to the similar procedures in step 4 to step 8 of compound I-1 under appropriate conditions recognized by one of ordinary skill in the art. ¹H NMR (400 MHz, DMSO-d₆) δ 7.54-7.48 (m, 2H), 7.46-7.38 (m, 2H), 7.17 (t, J=7.4 Hz, 1H), 7.12-7.04 (m, 4H), 6.38-6.22 (m, 3H), 6.14-6.04 (m, 1H), 5.70-5.61 (m, 1H), 4.80-4.67 (m, 1H), 4.33 (s, 1H), 4.22 (s, 1H), 4.04 (s, 1H), 3.93 (s, 1H), 2.75-2.57 (m, 4H). MS (ESI) m/e [M+1]⁺ 443.9.

### Compound I-20: (R)-1-(1-Acryloylpiperidin-3-yl)-5-amino-3-(4-isobutoxyphenyl)-1H-pyrazole-4-carboxamide

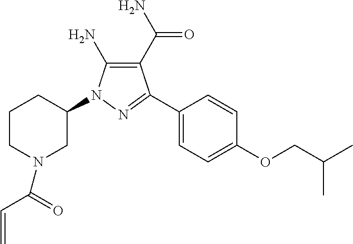 [SMILES: C=CC(=O)N1CCC[C@@H](n2nc(-c3ccc(OCC(C)C)cc3)c(C(N)=O)c2N)C1]

The desired product was prepared from (R)-tert-butyl 3-(5-amino-4-carbamoyl-3-(4-hydroxyphenyl)-1H-pyrazol-1-yl)piperidine-1-carboxylate and 1-bromo-2-methylpropane according to the similar procedures in step 4 to step 5 of compound I-16 under appropriate conditions recognized by one of ordinary skill in the art. ¹H NMR (400 MHz, DMSO-d₆) δ 7.38 (d, J=8.0 Hz, 2H), 7.00 (d, J=8.0 Hz, 2H), 6.93-6.75 (m, 1H), 6.42 (br s, 2H), 6.20-6.01 (m, 1H), 5.75-5.60 (m, 1H), 4.55-4.45 (m, 0.5H), 4.39-4.29 (m, 0.5H), 4.26-4.02 (m, 2H), 3.78 (d, J=6.8 Hz, 2H), 3.12-2.98 (m, 1H), 2.78-2.64 (m, 1H), 2.09-1.93 (m, 3H), 1.88-1.79 (m, 1H), 1.55-1.36 (m, 1H), 0.98 (d, J=6.8 Hz, 6H). MS (ESI) m/e [M+1]⁺ 412.0.

### Compound I-21: (R)-1-(1-Acryloylpiperidin-3-yl)-5-amino-3-(4-chloro-3-methoxyphenyl)-1H-pyrazole-4-carboxamide

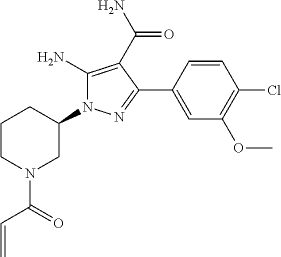 [SMILES: C=CC(=O)N1CCC[C@@H](n2nc(-c3ccc(Cl)c(OC)c3)c(C(N)=O)c2N)C1]

The desired product was prepared from 4-chloro-3-methoxybenzoic acid and (S)-tert-butyl 3-hydroxypiperidine-1-carboxylate according to the similar procedure in step 1 to step 8 of compound I-1 under appropriate conditions recognized by one of ordinary skill in the art. ¹H NMR (400 MHz, DMSO-d₆) δ 7.47 (d, J=8.0 Hz, 1H), 7.22 (d, J=6.0 Hz, 1H), 7.11-7.05 (m, 1H), 6.92-6.74 (m, 1H), 6.19-6.03 (m, 1H), 5.74-5.60 (m, 1H), 4.55-4.47 (m, 0.5H), 4.35-4.14 (m, 1.5H), 4.12-4.02 (m, 1H), 3.87 (s, 3H), 3.56-3.44 (m, 0.5H), 3.14-3.00 (m, 1H), 2.85-2.74 (m, 0.5H), 2.06-1.94 (m, 2H), 1.90-1.80 (m, 1H), 1.57-1.38 (m, 1H). MS (ESI) m/e [M+1]⁺403.9, 405.9.

### Example 2

### Synthesis of Compounds II-1 to II-10

### Compound II-1: 5-Acryloyl-3-amino-1-(4-phenoxyphenyl)-4,5,6,7-tetrahydro-1H-pyrrolo[3,2-c]pyridine-2-carboxamide

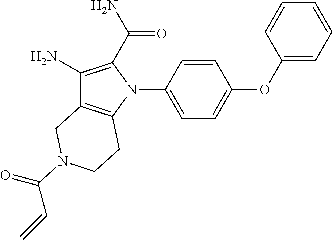 [SMILES: C=CC(=O)N1CCc2c(c(N)c(C(N)=O)n2-c2ccc(Oc3ccccc3)cc2)C1]

### Step 1: 2-(4-Phenoxyphenylamino)acetonitrile

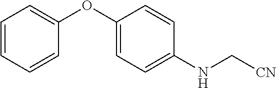 [SMILES: N#CCNc1ccc(Oc2ccccc2)cc1]

To a solution of 4-phenoxybenzenamine (12.5 g, 67.5 mmol) in 80 mL of THF was added DIPEA (14.14 mL, 81 mmol), followed by 2-bromoacetonitrile (8.9 g, 74.2 mmol). After stirring at 50° C. for 16 h, the reaction mixture was diluted with 120 mL of water and extracted with EA (80 mL×3), the combined organic phases were dried over Na₂SO₄, filtered and concentrated under reduced pressure. The crude residue was purified by chromatograph on a silica gel, eluting with EA/PE=¼ to give 11.6 g (76.8%) of 2-(4-phenoxyphenylamino)acetonitrile as a gray solid. ¹H NMR (400 MHz, DMSO-d₆) δ 7.34-7.30 (m, 2H), 7.09-7.01 (m, 1H), 6.96-6.86 (m, 4H), 6.80-6.73 (m, 2H), 6.22 (t, J=6.8 Hz, 1H), 4.26 (d, J=6.8 Hz, 2H). MS (ESI) m/e [M+1]⁺ 225.1.

### Step 2: Isopropyl 3-aminopropanoate hydrochloride

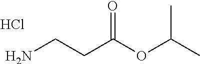

3-(tert-Butoxycarbonylamino)propanoic acid (100 g, 0.53 mol) was dissolved in 500 mL of 5N HCl in isopropanol. After stirring at RT for 16 h, the reaction mixture was concentrated under reduced pressure. The residue was washed with 200 mL of EA and filtered to give 80 g (90.2%) of the desired compound as a white solid. MS (ESI) m/e [M+1]⁺ 132.1.

### Step 3: Ethyl 3-(2-cyanoethylamino)propanoate

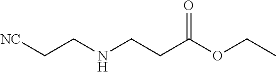

To a solution of isopropyl 3-aminopropanoate hydrochloride (80 g, 0.48 mol) in 500 mL of EtOH was added acrylonitrile (28 g, 0.53 mol), followed by NaOH (42.3 g, 1.06 mol). After stirring at 50° C. for 5 h, the reaction mixture was cooled to RT. 20 mL of con.H₂SO₄ was added slowly until pH=1, the mixture was heated to 50° C. for 16 h. After cooling down to RT, the mixture was basified with sat. NaHCO₃ until pH=8 and extracted with EA (500 mL×3). The combined organic phases were dried over Na₂SO₄, filtered and concentrated under reduced pressure to give 50 g (61%) of crude product as a yellow oil, which was used in the next step without further purification. MS (ESI) m/e [M+1]⁺ 171.0.

### Step 4: Ethyl 3-(tert-butoxycarbonyl(2-cyanoethyl)amino)propanoate

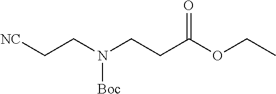 [SMILES: CCOC(=O)CCN(CCC#N)C(=O)OC(C)(C)C]

To a solution of ethyl 3-(2-cyanoethylamino)propanoate (24 g, 141 mmol) in 200 mL of DCM was added tert-butyl dicarbonate (47.9 g, 212 mmol). After stirring at RT for 16 hours, the reaction mixture was concentrated under reduced pressure. The crude residue was purified by chromatograph on 500 g of silica gel (eluting with EA/PE=from 1/10 to ¼) to give 22 g (57.9%) of the title compound as a colorless oil. ¹H NMR (400 MHz, CDCl₃-d₁) δ 4.20-4.08 (m, 2H), 3.59-3.50 (m, 4H), 2.70-2.50 (m, 4H), 1.47 (s, 9H), 1.33-1.19 (m, 3H). MS (ESI) m/e [M+23]⁺ 293.0.

### Step 5: tert-Butyl 3-cyano-4-oxopiperidine-1-carboxylate

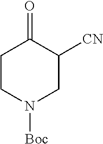 [SMILES: CC(C)(C)OC(=O)N1CCC(=O)C(C#N)C1]

To a solution of ethyl 3-(tert-butoxycarbonyl(2-cyanoethyl)amino)propanoate (17.3 g, 64.0 mmol) in 200 mL of toluene was added NaH (60%, 3.84 g, 96.0 mmol), the resulting mixture was heated to reflux for 3 h. After cooling down to RT, the mixture was diluted with 200 mL of water, subsequently acidified with 1N HCl solution until pH=3. The aqueous phase was extracted with EA (200 mL×3). The combined organic phases were dried over Na₂SO₄, filtered and concentrated under reduced pressure. The residue was purified by chromatograph on a silica gel, eluting with EA/PE=¼ to give 7.8 g (51.6%) of tert-butyl 3-cyano-4-oxopiperidine-1-carboxylate as a white solid. ¹H NMR (400 MHz, DMSO-d₆) δ 10.87 (s, 1H), 3.89 (s, 2H), 3.46 (t, J=5.8 Hz, 2H), 2.28 (t, J=5.8 Hz, 2H).

### Step 6: tert-Butyl 3-cyano-4-((cyanomethyl)(4-phenoxyphenyl)amino)-5,6-dihydropyridine-1(2H)-carboxylate

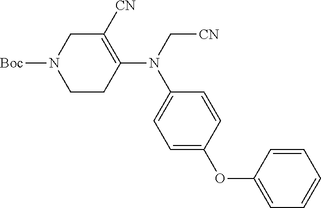 [SMILES: CC(C)(C)OC(=O)N1CCC(N(CC#N)c2ccc(Oc3ccccc3)cc2)=C(C#N)C1]

To a solution of tert-butyl 3-cyano-4-oxopiperidine-1-carboxylate (13.6 g, 61 mmol) in 50 mL of dry toluene was added 2-(4-phenoxyphenylamino)acetonitrile (11.38 g, 51 mmol), followed by 4-methylbenzenesulfonic acid (1.36 g, 6.1 mmol). The resulting mixture was heated to reflux with a Dean-Stark for 5 h. After cooling down to RT, the mixture was washed with 50 mL of sat. NaHCO₃ solution. The organic phase was collected, the aqueous phase was further extracted with EA (30 mL×3). The combined organic phases were dried over Na₂SO₄, filtered and concentrated under reduced pressure. The crude residue was purified by chromatograph on 300 g of silica gel (eluting with EA/PE=from ⅙ to ¼) to give 7.3 g (27.8%) of the desired compound as a gray solid. ¹H NMR (400 MHz, DMSO-d₆) δ 7.41-7.34 (m, 2H), 7.28-7.23 (m, 2H), 7.16-7.10 (m, 1H), 7.10-7.05 (m, 2H), 7.04-6.99 (m, 2H), 4.86 (s, 2H), 3.97 (s, 2H), 3.50 (t, J=5.6 Hz, 2H), 2.44 (t, J=5.6 Hz, 2H), 1.42 (s, 9H). MS (ESI) m/e [M+23]⁺ 430.9.

### Step 7: tert-Butyl 3-amino-2-cyano-1-(4-phenoxyphenyl)-6,7-dihydro-1H-pyrrolo[3,2-c]pyridine-5(4H)-carboxylate

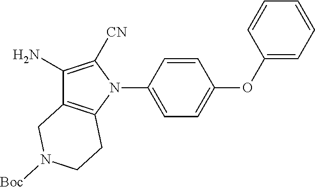 [SMILES: CC(C)(C)OC(=O)N1CCc2c(c(N)c(C#N)n2-c2ccc(Oc3ccccc3)cc2)C1]

To a solution of tert-butyl 3-cyano-4-((cyanomethyl)(4-phenoxyphenyl)amino)-5,6-dihydropyridine-1(2H)-carboxylate (7.3 g, 16.9 mmol) in 100 mL of t-BuOH was added t-BuONa (1.96 g, 20.3 mmol), the resulting mixture was heated to 100° C. for 1 h. After cooling down to RT, the solvent was removed under reduced pressure. The residue was diluted with 50 mL of water and extracted with EA (50 mL×3). The combined organic phases were dried over Na₂SO₄, filtered and concentrated under reduced pressure. The crude residue was purified by chromatograph on 100 g of silica gel (eluting with EA/PE=¼) to give 3.5 g (47.9%) of the desired compound as a gray solid. ¹H NMR (400 MHz, DMSO-d₆) δ 7.47-7.40 (m, 2H), 7.36 (d, J=8.6 Hz, 2H), 7.20 (t, J=7.4 Hz, 1H), 7.13-7.04 (m, 4H), 5.32 (s, 2H), 4.22 (s, 2H), 3.52 (t, J=5.6 Hz, 2H), 2.44 (t, J=5.6 Hz, 2H), 1.43 (s, 9H). MS (ESI) m/e [M+23]⁺430.9.

### Step 8: 3-Amino-1-(4-phenoxyphenyl)-4,5,6,7-tetrahydro-1H-pyrrolo[3,2-c]pyridine-2-carbonitrile dihydrochloride

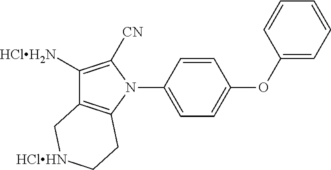

tert-Butyl 3-amino-2-cyano-1-(4-phenoxyphenyl)-6,7-dihydro-1H-pyrrolo[3,2-c]pyridine-5(4H)-carboxylate (550 mg, 1.28 mmol) was dissolved in 20 mL of 5N HCl in isopropanol. After stirring at RT for 5 h, the reaction mixture was concentrated under reduced pressure to afford 460 mg (89.3%) of crude product as a grey solid, which was used in the next step without further purification. ¹H NMR (400 MHz, DMSO-d₆) δ 9.62 (br s, 2H), 7.50-7.42 (m, 2H), 7.37-7.32 (m, 2H), 7.25-7.19 (m, 1H), 7.16-7.08 (m, 4H), 6.43 (br s, 3H), 3.98 (t, J=5.8 Hz, 2H), 3.35-3.25 (m, 2H), 2.69 (t, J=5.8 Hz, 2H). MS (ESI) m/e [M+23]⁺ 330.9.

### Step 9: 3-Amino-1-(4-phenoxyphenyl)-4,5,6,7-tetrahydro-1H-pyrrolo[3,2-c]pyridine-2-carboxamide

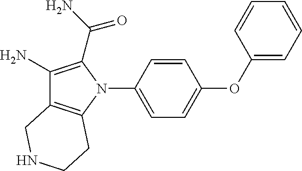 [SMILES: NC(=O)c1c(N)c2c(n1-c1ccc(Oc3ccccc3)cc1)CCNC2]

20 mL of H₃PO₄ was heated to 130° C. 3-Amino-1-(4-phenoxyphenyl)-4,5,6,7-tetrahydro-1H-pyrrolo[3,2-c]pyridine-2-carbonitrile dihydrochloride (460 mg, 1.14 mmol) was added and stirred at this temperature for 10 min After cooling down to RT, the mixture was basified with NaHCO₃ until pH=8. The aqueous phase was extracted with EA (100 mL×3). The combined organic phases were dried over Na₂SO₄, filter and concentrated under reduced pressure to give 340 mg (86%) of the desired product as a gray solid, which was used in the next step without further purification. ¹H NMR (400 MHz, DMSO-d₆) δ 7.47-7.39 (m, 2H), 7.25-7.16 (m, 3H), 7.11-7.03 (m, 4H), 3.57 (s, 2H), 2.88 (t, J=5.4 Hz, 2H), 2.20 (t, J=5.4 Hz, 2H). MS (ESI) m/e [M+23]⁺ 348.9.

### Step 10: 5-Acryloyl-3-amino-1-(4-phenoxyphenyl)-4,5,6,7-tetrahydro-1H-pyrrolo[3,2-c]pyridine-2-carboxamide

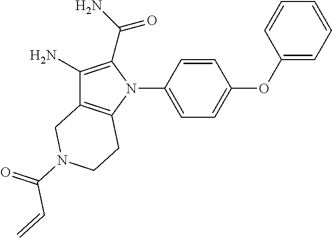 [SMILES: C=CC(=O)N1CCc2c(c(N)c(C(N)=O)n2-c2ccc(Oc3ccccc3)cc2)C1]

To a solution of 3-amino-1-(4-phenoxyphenyl)-4,5,6,7-tetrahydro-1H-pyrrolo[3,2-c]pyridine-2-carboxamide (141 mg, 0.41 mmol) in 20 mL of DCM was added DIPEA (106 mg, 0.82 mmol), the mixture was cooled to 0° C. Then, acryloyl chloride (37 mg, 0.41 mmol) in 4 mL of DCM was added dropwise, the resulting mixture was stirred at 0° C. for 2 min. The reaction mixture was quenched with 40 mL of water. The aqueous phase was extracted with DCM (30 mL×3). The combined organic phases were dried over Na₂SO₄, filtered and concentrated under reduced pressure. The crude residue was purified by pre-HPLC to give 12 mg (7.27%) of the title compound as a white solid. ¹H NMR (400 MHz, DMSO-d₆ at 80° C.) δ 7.43-7.35 (m, 2H), 7.26-7.20 (m, 2H), 7.18-7.12 (m, 1H), 7.08-7.02 (m, 4H), 6.75 (dd, J=16.8, 10.4 Hz, 1H), 6.07 (dd, J=16.8, 2.1 Hz, 1H), 5.65 (dd, J=10.4, 2.1 Hz, 1H), 5.52 (br s, 2H), 5.23 (br s, 2H), 4.41 (s, 2H), 3.70 (t, J=5.8 Hz, 2H), 2.30 (t, J=5.8 Hz, 2H). MS (ESI) m/e [M+23]⁺ 402.9.

### Compound II-2: 5-(1-Acryloylpyrrolidin-3-yl)-3-amino-1-(4-phenoxyphenyl)-4,5,6,7-tetrahydro-1H-pyrrolo[3,2-c]pyridine-2-carboxamide

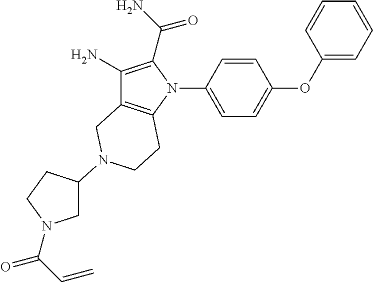 [SMILES: C=CC(=O)N1CCC(N2CCc3c(c(N)c(C(N)=O)n3-c3ccc(Oc4ccccc4)cc3)C2)C1]

### Step 1: tert-Butyl 3-(3-amino-2-carbamoyl-1-(4-phenoxyphenyl)-6,7-dihydro-1H-pyrrolo[3,2-c]pyridin-5(4H)-yl)pyrrolidine-1-carboxylate

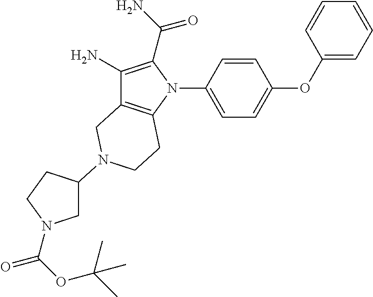 [SMILES: CC(C)(C)OC(=O)N1CCC(N2CCc3c(c(N)c(C(N)=O)n3-c3ccc(Oc4ccccc4)cc3)C2)C1]

To a solution of 3-amino-1-(4-phenoxyphenyl)-4,5,6,7-tetrahydro-1H-pyrrolo[3,2-c]pyridine-2-carboxamide (112.8 mg, 0.32 mmol) in 20 mL of MeOH was added tert-butyl 3-oxopyrrolidine-1-carboxylate (59.2 mg, 0.32 mmol), followed by HOAc (0.1 mL). The mixture was stirred at RT for 1 h, NaCNBH₃ (40 mg, 0.64 mmol) was added. The resulting mixture was stirred at RT for 16 h and quenched with 40 mL of sat. NH₄Cl solution, then extracted with EA (30 mL×3). The combined organic phases were dried over Na₂SO₄, filtered and concentrated under reduced pressure. The crude residue was purified by chromatograph on a silica gel, eluting with EA/PE=¼ to give 58 mg (35.2%) of desired compound as a gray solid. MS (ESI) m/e [M+1]⁺517.9.

### Step 2: 3-Amino-1-(4-phenoxyphenyl)-5-(pyrrolidin-3-yl)-4,5,6,7-tetrahydro-1H-pyrrolo[3,2-c]pyridine-2-carboxamide dihydrochloride

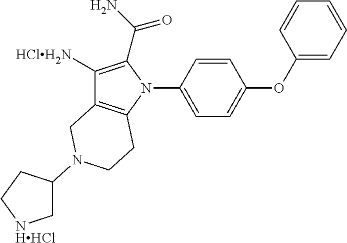

tert-Butyl 3-(3-amino-2-carbamoyl-1-(4-phenoxyphenyl)-6,7-dihydro-1H-pyrrolo[3,2-c]pyridin-5(4H)-yl)pyrrolidine-1-carboxylate (58 mg, 0.11 mmol) was dissolved in 20 mL of 5N HCl in isopropanol. The resulting mixture was stirred at RT for 16 h and concentrated under reduced pressure. The crude residue was washed with 10 mL of EA and filtered to afford 43 mg (81.1%) of the desired compound as a gray solid. MS (ESI) m/e [M+1]⁺ 417.9.

### Step 3: 5-(1-Acryloylpyrrolidin-3-yl)-3-amino-1-(4-phenoxyphenyl)-4,5,6,7-tetrahydro-1H-pyrrolo[3,2-c]pyridine-2-carboxamide

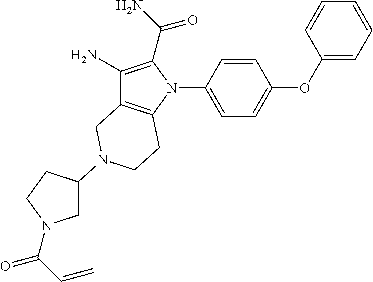 [SMILES: C=CC(=O)N1CCC(N2CCc3c(c(N)c(C(N)=O)n3-c3ccc(Oc4ccccc4)cc3)C2)C1]

To a solution of 3-amino-1-(4-phenoxyphenyl)-5-(pyrrolidin-3-yl)-4,5,6,7-tetrahydro-1H-pyrrolo[3,2-c]pyridine-2-carboxamide dihydrochloride (43 mg, 0.1 mmol) in 15 mL of DCM was added DIPEA (25.8 mg, 0.2 mmol). After cooling down to 0° C., acryloyl chloride (7.2 mg, 0.08 mmol) in 5 mL of DCM was added dropwise. The resulting mixture was stirred at this temperature for 2 min and quenched with 20 mL of water. The aqueous phase was extracted with DCM (20 mL×2). The combined organic phases were dried over Na₂SO₄, filtered and concentrated under reduced pressure. The crude residue was purified by pre-HPLC to give 14.2 mg (30.1%) of title compound as a white solid. ¹H NMR (400 MHz, DMSO-d₆ at 80° C.) δ 7.53-7.44 (m, 2H), 7.34-7.28 (m, 2H), 7.28-7.21 (m, 1H), 7.17-7.10 (m, 4H), 6.72-6.56 (m, 1H), 6.19 (dd, J=16.8, 2.2 Hz, 1H), 5.71 (dd, J=10.4, 2.2 Hz, 1H), 5.56 (br s, 2H), 3.97-3.53 (m, 2H), 3.45 (s, 2H), 3.30-3.05 (m, 3H), 2.87-2.65 (m, 2H), 2.41-2.32 (m, 2H), 2.31-2.13 (m, 1H), 2.04-1.76 (m, 1H). MS (ESI) m/e [M+1]⁺ 471.9.

### Compound II-3: 5-(2-Acrylamidoethyl)-3-amino-1-(4-phenoxyphenyl)-4,5,6,7-tetrahydro-1H-pyrrolo[3,2-c]pyridine-2-carboxamide

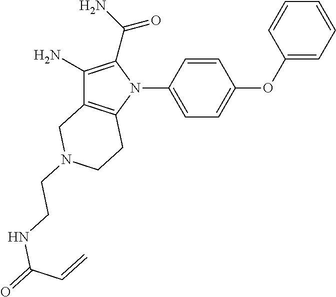 [SMILES: C=CC(=O)NCCN1CCc2c(c(N)c(C(N)=O)n2-c2ccc(Oc3ccccc3)cc2)C1]

The desired product was prepared from 3-amino-1-(4-phenoxyphenyl)-4,5,6,7-tetrahydro-1H-pyrrolo[3,2-c]pyridine-2-carboxamide and tert-butyl(2-oxoethyl)carbamate according to the similar procedure in step 1 to step 3 of compound II-2 under appropriate conditions recognized by one of ordinary skill in the art. ¹H NMR (400 MHz, DMSO-d₆) δ 9.71 (br s, 1H), 8.38 (s, 1H), 7.48-7.41 (m, 2H), 7.29-7.24 (m, 2H), 7.24-7.18 (m, 1H), 7.12-7.05 (m, 4H), 6.22 (dd, J=17.2, 9.8 Hz, 1H), 6.13 (dd, J=17.2, 2.4 Hz, 1H), 5.67 (dd, J=9.8, 2.4 Hz, 1H), 5.94 (br s, 2H), 4.44-4.34 (m, 1H), 4.08-4.96 (m, 1H), 3.75-3.66 (m, 1H), 3.63-3.50 (m, 2H), 3.45-3.25 (m, 3H), 2.61 (t, J=5.2, 2H). MS (ESI) m/e [M+1]⁺ 445.9.

### Compound II-4: 5-Acryloyl-3-amino-1-(4-(benzyloxy)phenyl)-4,5,6,7-tetrahydro-1H-pyrrolo[3,2-c]pyridine-2-carboxamide

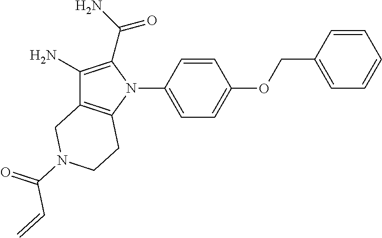 [SMILES: C=CC(=O)N1CCc2c(c(N)c(C(N)=O)n2-c2ccc(OCc3ccccc3)cc2)C1]

The desired product was prepared from 4-(benzyloxy)aniline, 2-bromoacetonitrile and tert-butyl 3-cyano-4-oxopiperidine-1-carboxylate according to the similar procedure in step 1 and step 6 to step 10 of compound II-1 under appropriate conditions recognized by one of ordinary skill in the art. ¹H NMR (400 MHz, DMSO-d₆ at 80° C.) δ 7.46 (d, J=7.4 Hz, 2H), 7.40 (d, J=7.4 Hz, 1H), 7.38 (d, J=7.4 Hz, 1H), 7.36-7.30 (m, 1H), 7.19 (d, J=8.6 Hz, 2H), 7.10 (d, J=8.6 Hz, 2H), 6.76 (dd, J=16.8, 10.2 Hz, 1H), 6.09 (d, J=16.8 Hz, 1H), 5.66 (d, J=10.2 Hz, 1H), 5.43 (s, 2H), 5.26 (s, 2H), 5.14 (s, 2H), 4.42 (s, 2H), 3.70 (t, J=5.6 Hz, 2H), 2.27 (t, J=5.6 Hz, 2H). MS (ESI) m/e [M+1]⁺ 416.9.

### Compound II-5: 5-Acryloyl-3-amino-1-(4-phenoxyphenyl)-1,4,5,6-tetrahydropyrrolo[3,4-b]pyrrole-2-carboxamide

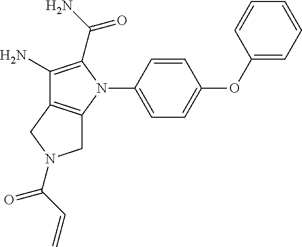 [SMILES: C=CC(=O)N1Cc2c(N)c(C(N)=O)n(-c3ccc(Oc4ccccc4)cc3)c2C1]

The desired product was prepared from 2-(4-phenoxyphenylamino)acetonitrile and tert-butyl 3-cyano-4-oxopyrrolidine-1-carboxylate according to the similar procedure in step 6 to step 10 of compound II-1 under appropriate conditions recognized by one of ordinary skill in the art. ¹H NMR (400 MHz, DMSO-d₆) δ 7.46-7.37 (m, 2H), 7.33-7.25 (m, 2H), 7.21-7.14 (m, 1H), 7.12-7.02 (m, 4H), 6.64-6.51 (m, 1H), 6.25-6.13 (m, 1H), 6.00 (br s, 2H), 5.80-5.64 (m, 1H), 5.53 (s, 2H), 4.65 (s, 1H), 4.60 (s, 1H), 4.38 (s, 2H). MS (ESI) m/e [M+1]⁺ 388.9.

### Compound II-6: 5-Acryloyl-3-amino-1-(4-(4-(tert-butyl)phenoxy)phenyl)-1,4,5,6-tetrahydropyrrolo[3,4-b]pyrrole-2-carboxamide

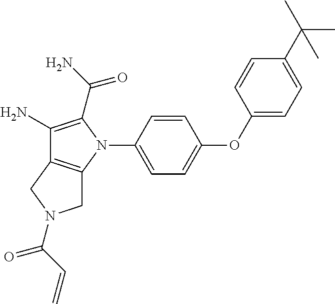 [SMILES: C=CC(=O)N1Cc2c(N)c(C(N)=O)n(-c3ccc(Oc4ccc(C(C)(C)C)cc4)cc3)c2C1]

5-Acryloyl-3-amino-1-(4-(4-(tert-butyl)phenoxy)phenyl)-1,4,5,6-tetrahydropyrrolo[3,4-b]pyrrole-2-carboxamide was obtained as a byproduct in the preparation of compound II-5. ¹H NMR (400 MHz, DMSO-d₆) δ 7.43 (dd, J=8.6, 2.0 Hz, 2H), 7.28 (dd, J=8.6, 2.0 Hz, 2H), 7.09-6.95 (m, 4H), 6.64-6.51 (m, 1H), 6.25-6.11 (m, 1H), 5.99 (br s, 2H), 5.80-5.63 (m, 1H), 5.53 (br s, 2H), 4.64 (s, 1H), 4.60 (s, 1H), 4.37 (s, 2H), 1.29 (s, 9H). MS (ESI) m/e [M+1]⁺ 445.0.

### Compound II-7: 5-Acrylamido-3-amino-1-(4-phenoxyphenyl)-1H-indole-2-carboxamide

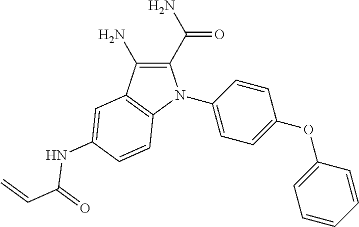 [SMILES: C=CC(=O)Nc1ccc2c(c1)c(N)c(C(N)=O)n2-c1ccc(Oc2ccccc2)cc1]

### Step 1: 5-Nitro-2-((4-phenoxyphenyl)amino)benzonitrile

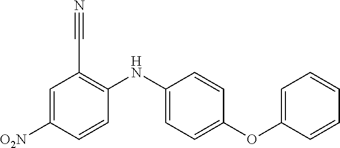 [SMILES: N#Cc1cc([N+](=O)[O-])ccc1Nc1ccc(Oc2ccccc2)cc1]

To a solution of 2-fluoro-5-nitrobenzonitrile (5.0 g, 30 mmol) in DMF (50 mL) was added TEA (6.1 g, 60 mmol), and 4-phenoxyaniline (7.4 g, 30 mmol), the reaction was warmed to 110° C. for 16 h. After cooling down to RT, the reaction was concentrated under reduced pressure. The residue was dissolved in DCM (100 mL) and washed with water (20 mL×2), dried over Na₂SO₄, filtered, concentrated and purified by column chromatography on silica gel (200-300 mesh, PE/EA=2/1) to give the product about 5.0 g (50.4%). MS (ESI) m/e [M+1]⁺332.1.

### Step 2: 3-Amino-5-nitro-1-(4-phenoxyphenyl)-1H-indole-2-carbonitrile

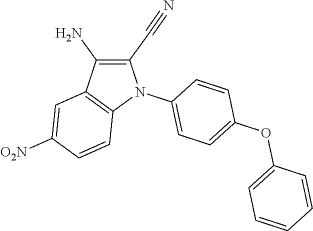 [SMILES: N#Cc1c(N)c2cc([N+](=O)[O-])ccc2n1-c1ccc(Oc2ccccc2)cc1]

To a solution of 5-nitro-2-((4-phenoxyphenyl)amino)benzonitrile (33 mg, 0.10 mmol) in DMF (5.0 mL) was added K₂CO₃ (28 mg, 0.20 mmol), AgNO₃ (17 mg, 0.10 mmol) and 2-bromoacetonitrile (18 mg, 0.15 mmol), the reaction was stirred at RT for 16 h, then heated to 100° C. for 2 h. After cooling down to RT, the reaction mixture was poured to water (25 mL), the aqueous was extracted with EA (10 mL×3). The combined organic phases were washed with brine (10 mL), dried over Na₂SO₄, filtered, concentrated and purified by pre-TLC (PE/EA=3/1), got the product as a white solid about 20 mg (55%). ¹H NMR (400 MHz, DMSO-d₆) δ 9.08 (d, J=2.0 Hz, 1H), 8.15 (dd, J=2.0, 9.2 Hz, 1H), 7.58-7.54 (m, 2H), 7.50-7.44 (m, 2H), 7.29 (d, J=9.2 Hz, 1H), 7.26-7.14 (m, 5H), 6.66 (br s, 2H). MS (ESI) m/e [M+1]⁺ 371.1.

### Step 3: 3-Amino-5-nitro-1-(4-phenoxyphenyl)-1H-indole-2-carboxamide

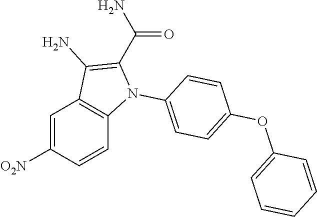 [SMILES: NC(=O)c1c(N)c2cc([N+](=O)[O-])ccc2n1-c1ccc(Oc2ccccc2)cc1]

To a solution of 3-amino-5-nitro-1-(4-phenoxyphenyl)-1H-indole-2-carbonitrile (300 mg, 0.81 mmol) in DMSO (6.0 mL) and EtOH (6.0 mL) was added NaOH in water (5.0 M, 3.0 mL) and H₂O₂ (3.0 mL) The reaction was stirred at RT for about 30 min and concentrated under reduced pressure to remove EtOH. The residue was suspended in water (50 mL), and extracted with EA (20 mL×4). The combined organic phases were washed with brine (10 mL), dried over Na₂SO₄, filtered and concentrated. The residue was suspended in EA (10 mL). The solid was washed with EA and dried under reduced pressure to give 150 mg (47.7%) of desired product as a red solid. ¹H NMR (DMSO-d₆) δ 9.04 (d, J=2.4 Hz, 1H), 8.07 (dd, J=2.4, 9.2 Hz, 1H), 7.48-7.40 (m, 4H), 7.24-7.10 (m, 6H), 6.22 (br s, 2H). MS (ESI) m/e [M+1]⁺ 389.1.

### Step 4: 3,5-Diamino-1-(4-phenoxyphenyl)-1H-indole-2-carboxamide

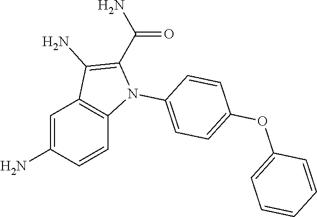 [SMILES: NC(=O)c1c(N)c2cc(N)ccc2n1-c1ccc(Oc2ccccc2)cc1]

To a solution of 3-amino-5-nitro-1-(4-phenoxyphenyl)-1H-indole-2-carboxamide (100 mg, 0.26 mmol) in MeOH (10 mL) was added 10% w/w Pd/C (40 mg). The reaction was exchanged with H₂ for three times and stirred at RT under H₂ atmosphere for about 3 h. Filtered and washed with MeOH (20 mL), the filtrate was concentrated under reduced pressure to give the crude product about 50 mg (54.2%) as a yellow solid. ¹H NMR (400 MHz, DMSO-d₆) δ 7.45-7.40 (m, 2H), 7.28-7.23 (m, 2H), 7.20-7.14 (m, 1H), 7.10-7.06 (m, 4H), 6.99-6.91 (m, 2H), 6.66 (dd, J=2.4, 8.8 Hz, 1H), 5.70 (br s, 2H), 4.71 (br s, 2H). MS (ESI) m/e [M+1]⁺ 359.1.

### Step 5: 5-Acrylamido-3-amino-1-(4-phenoxyphenyl)-1H-indole-2-carboxamide

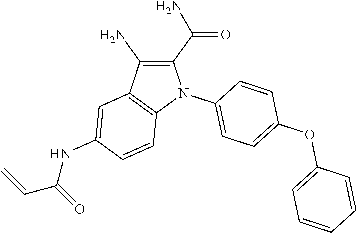 [SMILES: C=CC(=O)Nc1ccc2c(c1)c(N)c(C(N)=O)n2-c1ccc(Oc2ccccc2)cc1]

To a solution of 3,5-diamino-1-(4-phenoxyphenyl)-1H-indole-2-carboxamide (35 mg, 0.10 mmol) in DCM (10 mL) was added DIEA (26 mg, 0.20 mmol) and acryloyl chloride (9.0 mg, 0.10 mmol) at 0° C. After stirring at 0° C. for about 20 min, the reaction was quenched with water (5.0 mL). The aqueous was extracted with DCM (5.0 mL×3), the combined organic phases were washed with brine (20 mL), dried over Na₂SO₄, filtered, concentrated and purified by pre-TLC (PE/EA=¼), to give the product as a white solid about 6.0 mg (14.9%). ¹H NMR (400 MHz, CD₃OD-d₄) δ 8.13 (d, J=1.6 Hz, 1H), 7.43-7.33 (m, 5H), 7.20-7.02 (m, 6H), 6.46 (dd, J=10.0, 16.8 Hz, 1H), 6.36 (dd, J=2.0, 16.8 Hz, 1H), 5.77 (dd, J=2.0, 10.0 Hz, 1H). MS (ESI) m/e [M+1]⁺ 413.1.

### Compound II-8: 6-Acrylamido-3-amino-1-(4-phenoxyphenyl)-1H-indole-2-carboxamide

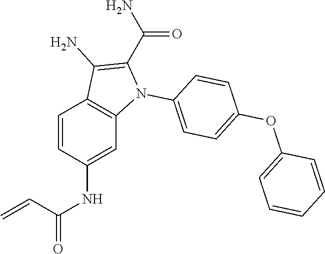 [SMILES: C=CC(=O)Nc1ccc2c(N)c(C(N)=O)n(-c3ccc(Oc4ccccc4)cc3)c2c1]

The desired product was prepared from 2-fluoro-4-nitrobenzonitrile and 4-phenoxyaniline according to the similar procedure in step 1 to step 5 of compound II-7 under appropriate conditions recognized by one of ordinary skill in the art. ¹H NMR (400 MHz, CD₃OD-d₄) δ 7.38-7.23 (m, 5H), 7.10-6.94 (m, 5H), 6.58 (dd, J=8.6, 1.8 Hz, 1H), 6.47 (dd, J=17.0, 10.2 Hz, 1H), 6.40-6.29 (m, 2H), 5.76 (d, J=10.2 Hz, 1H). MS (ESI) m/e [M+1]⁺ 413.1.

### Compound II-9: 3-Amino-1-(4-phenoxyphenyl)-4-(piperidin-4-yl)-1H-pyrrole-2-carboxamide

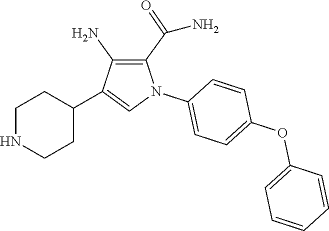 [SMILES: NC(=O)c1c(N)c(C2CCNCC2)cn1-c1ccc(Oc2ccccc2)cc1]

### Step 1: tert-Butyl 4-(1-cyano-2-oxoethyl)piperidine-1-carboxylate

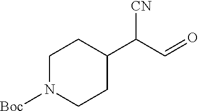 [SMILES: CC(C)(C)OC(=O)N1CCC(C(C#N)C=O)CC1]

t-BuOK (3 g, 26.7 mmol) was added portionwisely to a solution of tert-butyl 4-(cyanomethyl)piperidine-1-carboxylate (1 g, 4.46 mmol) in DMF (40 mL) at 5˜10° C. After addition, the mixture was stirred for 30 min. A solution of ethyl formate (2 g, 26.7 mmol) in DMF (10 mL) was added dropwise. Then the mixture was allowed to warm to RT and stirred at 35° C. for 12 h (overnight). 10 mL of water was added to quench the reaction. The solvent was removed under reduced pressure. 100 mL of water was added and extracted with EA (20 mL×2). The combined organic phases were dried over Na₂SO₄, filtered and concentrated to give 1 g (90%) of desired product as a yellow solid. ¹H NMR (400 MHz, CDCl₃-d₁) δ 8.88 (br s, 1H), 4.16-4.08 (m, 2H), 2.84-2.64 (m, 2H), 1.81-1.62 (m, 2H), 1.57-1.38 (m, 2H), 1.46 (s, 9H), 1.31-1.23 (m, 1H). MS (ESI) m/e [M+23]⁺ 275.1.

### Step 2: tert-Butyl 4-(1-cyano-2-((cyanomethyl)(4-phenoxyphenyl)amino)vinyl)piperidine-1-carboxylate

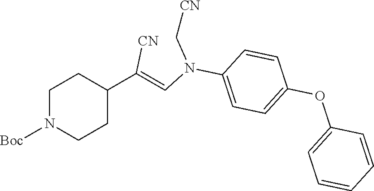 [SMILES: CC(C)(C)OC(=O)N1CCC(/C(C#N)=C/N(CC#N)c2ccc(Oc3ccccc3)cc2)CC1]

A mixture of tert-butyl 4-(1-cyano-2-oxoethyl)piperidine-1-carboxylate (281 mg, 1.115 mmol), 2-((4-phenoxyphenyl)amino)acetonitrile (250 mg, 1.115 mmol) and TsOH.H₂O (21 mg, 0.112 mmol) in toluene (40 mL) was heated at reflux for 4 h using a Dean-Stark. The mixture was filtered and added EA (50 mL×3). The organic phase was washed with brine, dried over Na₂SO₄, filtered, concentrated and purified by column chromatography on silica gel (200-300 mesh, PE/EA=10/1 to 3/1) to give 320 mg (63%) of desired product as a white solid. ¹H NMR (400 MHz, CDCl₃-d₁) δ 7.41-7.32 (m, 2H), 7.21-7.11 (m, 3H), 7.08-6.99 (m, 4H), 6.58 (s, 1H), 4.20 (s, 2H), 2.69 (t, J=11.8 Hz, 2H), 2.19 (t, J=11.8 Hz, 1H), 1.74 (d, J=12.2 Hz, 2H), 1.65-1.41 (m, 4H), 1.46 (s, 9H). MS (ESI) m/e [M+23]⁺ 481.2.

### Step 3: tert-Butyl 4-(4-amino-5-cyano-1-(4-phenoxyphenyl)-1H-pyrrol-3-yl)piperidine-1-carboxylate

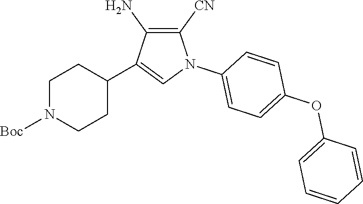 [SMILES: CC(C)(C)OC(=O)N1CCC(c2cn(-c3ccc(Oc4ccccc4)cc3)c(C#N)c2N)CC1]

To a solution of tert-butyl 4-(1-cyano-2-((cyanomethyl)(4-phenoxyphenyl)amino) vinyl)piperidine-1-carboxylate (30 mg, 0.065 mmol) in t-BuOH (10 mL) was added t-BuOK (8 mg, 0.072 mmol), the mixture was warmed to 80° C. and stirred for about 2 h. After cooling down to RT, to the mixture was added water (10 mL), then extracted with EA (10 mL×2). The combined organic phases were washed with brine (10 mL), dried over Na₂SO₄, filtered and concentrated. The residue was purified by Pre-TLC (DCM/EA=3/1) to give 10 mg (33%) of desired product as a colorless oil. ¹H NMR (400 MHz, CDCl₃d₁) δ 7.40-7.29 (m, 4H), 7.18-7.12 (m, 1H), 7.08-7.02 (m, 4H), 6.64 (s, 1H), 4.21 (d, J=11.6 Hz, 2H), 2.82 (t, J=12.5 Hz, 2H), 2.56 (t, J=11.6 Hz, 1H), 1.91 (d, J=12.5 Hz, 2H), 1.56-1.40 (m, 2H), 1.5 (s, 9H). MS (ESI) m/e [M+1]⁺ 459.2.

### Step 4: tert-Butyl 4-(4-amino-5-carbamoyl-1-(4-phenoxyphenyl)-1H-pyrrol-3-yl)piperidine-1-carboxylate

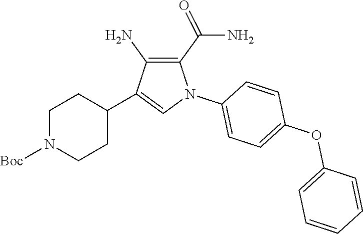 [SMILES: CC(C)(C)OC(=O)N1CCC(c2cn(-c3ccc(Oc4ccccc4)cc3)c(C(N)=O)c2N)CC1]

To a solution of tert-butyl 4-(4-amino-5-cyano-1-(4-phenoxyphenyl)-1H-pyrrol-3-yl)piperidine-1-carboxylate (180 mg, 0.39 mmol) in DMSO (5 mL) and EtOH (5 mL) was added NaOH (2.5 mL, 5 N) and H₂O₂ (2.5 mL), the reaction was stirred at RT for about 2 h. Concentrated under reduced pressure to remove EtOH, then added water (50 mL) to the reaction, the aqueous phase was extracted with EA (20 mL×3), The combined organic phases were washed with brine (10 mL), dried over Na₂SO₄, filtered and concentrated to give the crude product about 180 mg (96%). 7.5 mg of pure compound as a white solid was obtained by Pre-TLC (DCM/MeOH=10/1) and used for analysis. ¹H NMR (400 MHz, DMSO-d₆) δ 7.41 (t, J=8.2 Hz, 2H), 7.24 (d, J=8.8 Hz, 2H), 7.16 (t, J=7, 4 Hz, 1H), 7.07-7.01 (m, 4H), 6.67 (s, 1H), 6.11 (br s, 2H), 5.15 (br s, 2H), 4.05-3.97 (m, 2H), 2.95-2.70 (m, 2H), 2.63-2.55 (m, 1H), 1.86-1.79 (m, 2H), 1.40 (s, 9H), 1.37-1.24 (m, 2H). MS (ESI) m/e [M+1]⁺ 477.0.

### Step 5: 3-Amino-1-(4-phenoxyphenyl)-4-(piperidin-4-yl)-1H-pyrrole-2-carboxamide

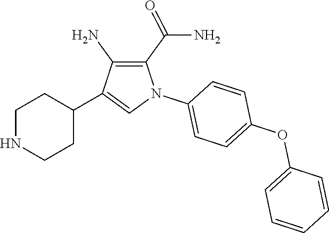 [SMILES: NC(=O)c1c(N)c(C2CCNCC2)cn1-c1ccc(Oc2ccccc2)cc1]

To a solution of tert-butyl 4-(4-amino-5-carbamoyl-1-(4-phenoxyphenyl)-1H-pyrrol-3-yl)piperidine-1-carboxylate (170 mg, 0.36 mmol) in DCM (10 mL) was added CF₃CO₂H (0.5 mL), the reaction was stirred at RT for about 16 h. Another batch of CF₃CO₂H (1.5 mL) was added to the reaction, then stirred for 2 h at RT. Concentrated under reduced pressure, the residue was purified by Pre-HPLC to give 130 mg (74%) of desired product as a white solid in CF₃CO₂H salt form. ¹H NMR (400 MHz, CD₃OD-d₄) a 7.42-7.32 (m, 4H), 7.19 (t, J=7.6 Hz, 1H), 7.12-7.02 (m, 4H), 6.95-6.88 (m, 1H), 3.52-3.45 (m, 2H), 3.15-3.06 (m, 2H), 2.96-2.86 (m, 1H), 2.21-2.13 (m, 21-1), 1.88-1.75 (m, 2H). MS (ESI) m/e [M+1]⁺ 377.0.

### Compound II-10: 4-(1-Acryloylpiperidin-3-yl)-3-amino-1-(4-phenoxyphenyl)-1H-pyrrole-2-carboxamide

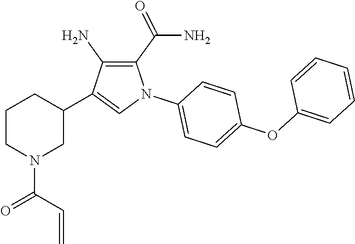 [SMILES: C=CC(=O)N1CCCC(c2cn(-c3ccc(Oc4ccccc4)cc3)c(C(N)=O)c2N)C1]

The desired product was prepared from tert-butyl 3-(cyanomethyl)piperidine-1-carboxylate (International Patent Publication No. WO 2011/111875) and 2-(4-phenoxyphenylamino)acetonitrile according to the similar procedures in step 1 to step 5 of compound 11-9 and step 8 of compound I-1 under appropriate conditions recognized by one of ordinary skill in the art. ¹H NMR (400 MHz, DMSO-d₆) δ 7.46-7.39 (m, 2H), 7.35-7.27 (m, 2H), 7.18 (t, J=7.4 Hz, 1H), 7.11-7.03 (m, 4H), 6.93 (s, 1H), 6.90-6.78 (m, 1H), 6.15-6.05 (m, 1H), 5.71-5.61 (m, 1H), 4.55-4.37 (m, 1H), 4.11-4.02 (m, 1H), 3.03 (t, J=12.0 Hz, 1H), 2.76-2.58 (m, 2H), 2.04-1.93 (m, 1H), 1.80-1.68 (m, 1H), 1.62-1.34 (m, 2H). MS (ESI) m/e [M+1]⁺430.9.

Btk Kinase Assay

Compounds disclosed herein were tested for inhibition of Btk kinase activity in an assay based on time-resolved fluorescence resonance energy transfer methodology. Recombinant Btk was pre-incubated with the compounds disclosed herein at room temperature for 1 hour in an assay buffer containing 50 mM Tris pH7.4, 10 mM MgCl₂, 2 mM MnCl₂, 0.1 mM EDTA, 1 mM DTT, 20 nM SEB, 0.1% BSA, 0.005% tween-20. The reactions were initiated by the addition of ATP (at the concentration of ATP Km) and peptide substrate (Biotin-AVLESEEELYSSARQ-NH2). After incubating at room temperature for 1 h, an equal volume of stop solution containing 50 mM HEPES pH7.0, 800 mM KF, 20 mM EDTA, 0.1% BSA, Eu cryptate-conjugated p-Tyr66 antibody and streptavidin-labeled XL665 was added to stop the reaction. Plates were further incubated at room temperature for 1 hour, and then the TR-FRET signals (ex337 nm, em 620 nm/665 nm) were read on BMG PHERAstar FS instrument. The residual enzyme activity in presence of increasing concentrations of compounds was calculated based on the ratio of fluorescence at 615 nm to that at 665 nm. The IC₅₀ for each compound was derived from fitting the data to the four-parameter logistic equation by Graphpad Prism software.

BtkpY223 Cellular Assay

Btk pY223 cellular assay is a HTRF based assay intended to determine the endogenous levels of phosphorylated Btk at Tyr223. Phosphorylated Tyr223 is necessary for full activation of Btk. The assay was performed in Ramos cells (CRL-1596, ATCC) with a Btk pY223 assay kit (63IDC000, Cisbio).

Briefly, Ramos cells were serum starved in 0.5% FBS-containing RPMI1640 for 2 hours. Following starvation, the cells were incubated with compounds to be detected at various concentrations in a CO2 incubator for 1 hour. After incubation, cells were stimulated with 1 mM pervanadate (PV) or Na₃VO₄ (OV) for 20 min. Then, the cells were spinned down and lysed with 1× lysis buffer at RT for 10 min (4× lysis buffer supplied in the kit). During the incubation, 1× antibody mix was prepared by diluting anti-Btk-d2 and anti-pBtk-K in detection buffer (supplied in the kit). 2 ul/well of 1× antibody mixture was dispensed into the OptiPlate-384 assay plate (6005620, Perkin Elmer). After that, 18 ul of cell lysate was transferred to the assay plate pre-loaded with antibody solution. After mixing gently and spinning briefly, the plate was sealed up and kept in dark at RT for 18 hours. The fluorescence emission was measured at two different wavelengths (665 nm and 620 nm) on a compatible HTRF reader (PHERAstar FS, BMG). The potency of compounds was calculated basing on the inhibition of ratio between signal intensities at 665 nm and 620 nm IC50 values were calculated with GraphPad Prism software using the sigmoidal dose-response function.

Representative compounds as disclosed herein were tested and found to inhibit Btk and autophosphorylation of Btk at Tyr-223 with IC50 values ranging from subnanomolar to 10 micromolar.

TABLE I
Assay data for representative compounds

| Com- | | 1050 (nM) | |
|---|---|---|---|
| pound | | | Btk |
| No. | Structure | Btk | pY223 |
| I-1 | 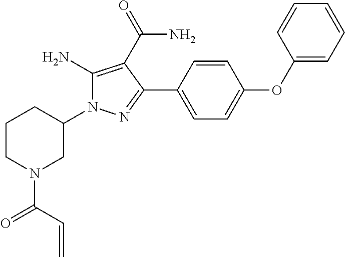 [SMILES: C=CC(=O)N1CCCC(n2nc(-c3ccc(Oc4ccccc4)cc3)c(C(N)=O)c2N)C1] | 0.52 | 3.5 |
| I-2 | 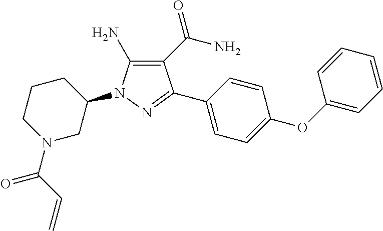 [SMILES: C=CC(=O)N1CCC[C@@H](n2nc(-c3ccc(Oc4ccccc4)cc3)c(C(N)=O)c2N)C1] | 0.17 | 1.0 |
| I-3 | 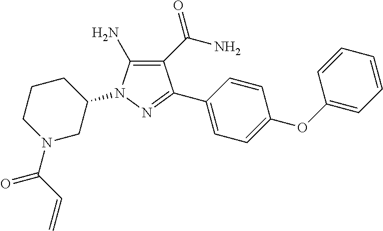 [SMILES: C=CC(=O)N1CCC[C@H](n2nc(-c3ccc(Oc4ccccc4)cc3)c(C(N)=O)c2N)C1] | 0.80 | 3.8 |
| I-4 | 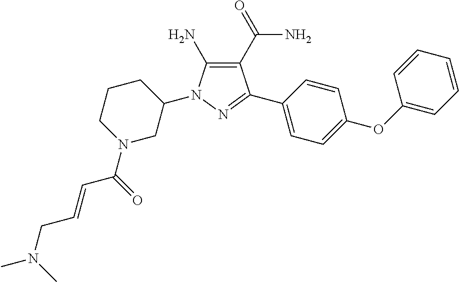 [SMILES: CN(C)C/C=C/C(=O)N1CCCC(n2nc(-c3ccc(Oc4ccccc4)cc3)c(C(N)=O)c2N)C1] | 0.56 | 3.6 |
| I-5 | 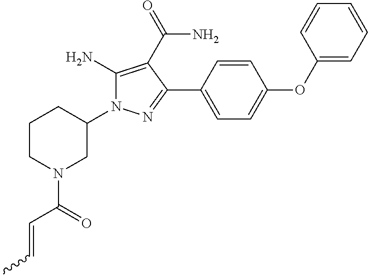 | 2.50 | 42.6 |
| I-6 | 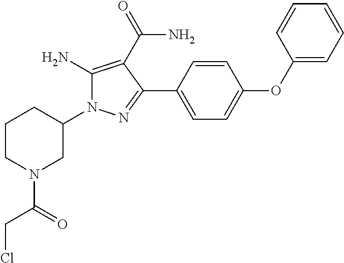 [SMILES: NC(=O)c1c(-c2ccc(Oc3ccccc3)cc2)nn(C2CCCN(C(=O)CCl)C2)c1N] | 0.22 | 10.4 |
| I-7 | 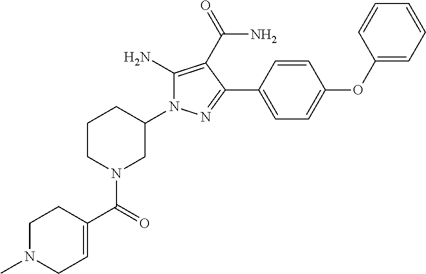 [SMILES: CN1CC=C(C(=O)N2CCCC(n3nc(-c4ccc(Oc5ccccc5)cc4)c(C(N)=O)c3N)C2)CC1] | 14 | n.d. |
| I-8 | 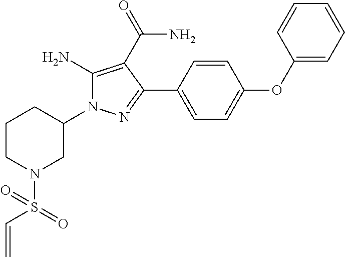 [SMILES: C=CS(=O)(=O)N1CCCC(n2nc(-c3ccc(Oc4ccccc4)cc3)c(C(N)=O)c2N)C1] | 0.23 | n.d. |
| I-9 | 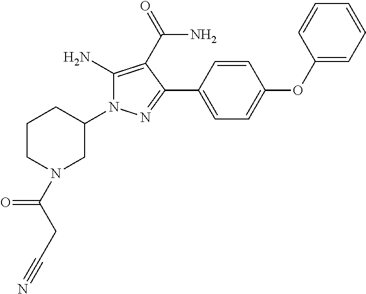 [SMILES: N#CCC(=O)N1CCCC(n2nc(-c3ccc(Oc4ccccc4)cc3)c(C(N)=O)c2N)C1] | 22 | n.d. |
| I-10 | 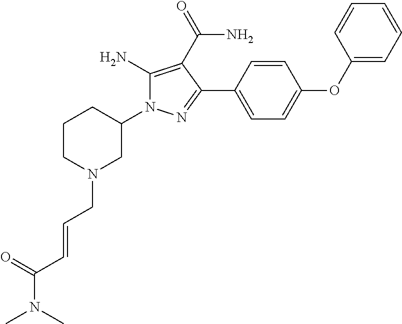 [SMILES: CN(C)C(=O)/C=C/CN1CCCC(n2nc(-c3ccc(Oc4ccccc4)cc3)c(C(N)=O)c2N)C1] | 18 | n.d. |
| I-11 | 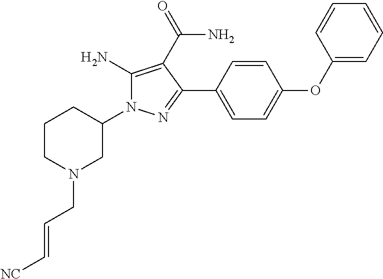 [SMILES: N#C/C=C/CN1CCCC(n2nc(-c3ccc(Oc4ccccc4)cc3)c(C(N)=O)c2N)C1] | 7.6 | n.d. |
| I-12 | 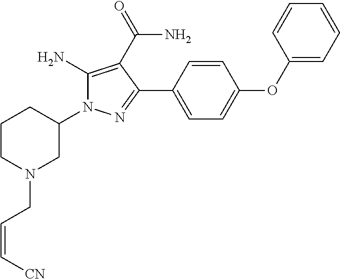 [SMILES: N#C/C=C\CN1CCCC(n2nc(-c3ccc(Oc4ccccc4)cc3)c(C(N)=O)c2N)C1] | 18 | n.d. |
| I-13 | 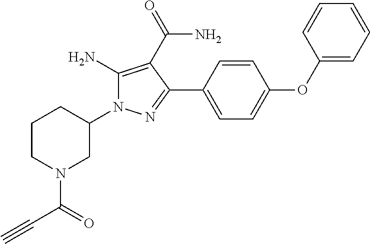 [SMILES: C#CC(=O)N1CCCC(n2nc(-c3ccc(Oc4ccccc4)cc3)c(C(N)=O)c2N)C1] | 0.21 | 7.5 |
| I-14 | 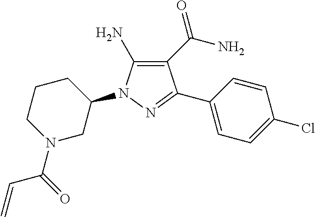 [SMILES: C=CC(=O)N1CCC[C@@H](n2nc(-c3ccc(Cl)cc3)c(C(N)=O)c2N)C1] | 0.54 | 3.3 |
| I-15 | 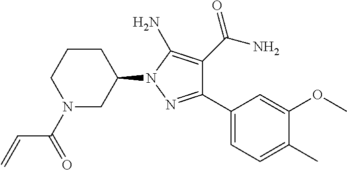 [SMILES: C=CC(=O)N1CCC[C@@H](n2nc(-c3ccc(C)c(OC)c3)c(C(N)=O)c2N)C1] | 0.24 | 0.3 |
| I-16 | 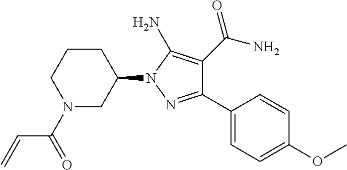 [SMILES: C=CC(=O)N1CCC[C@@H](n2nc(-c3ccc(OC)cc3)c(C(N)=O)c2N)C1] | 1.4 | 17.7 |
| I-17 | 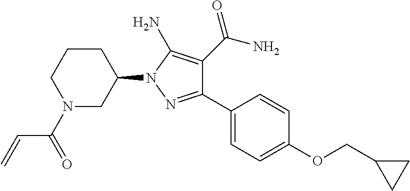 [SMILES: C=CC(=O)N1CCC[C@@H](n2nc(-c3ccc(OCC4CC4)cc3)c(C(N)=O)c2N)C1] | 0.92 | n.d. |
| I-18 | 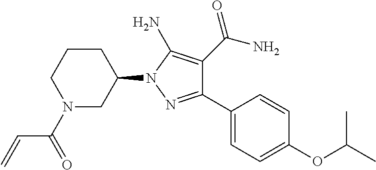 [SMILES: C=CC(=O)N1CCC[C@@H](n2nc(-c3ccc(OC(C)C)cc3)c(C(N)=O)c2N)C1] | 2.4 | n.d. |
| I-19 | 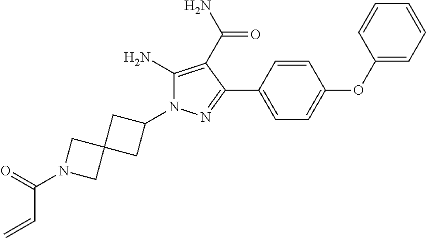 [SMILES: C=CC(=O)N1CC2(CC(n3nc(-c4ccc(Oc5ccccc5)cc4)c(C(N)=O)c3N)C2)C1] | 0.15 | n.d. |
| I-20 | 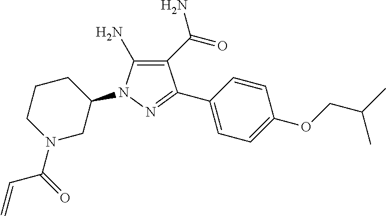 [SMILES: C=CC(=O)N1CCC[C@@H](n2nc(-c3ccc(OCC(C)C)cc3)c(C(N)=O)c2N)C1] | 0.49 | n.d. |
| I-21 | 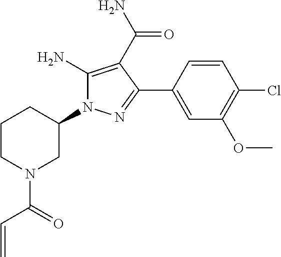 [SMILES: C=CC(=O)N1CCC[C@@H](n2nc(-c3ccc(Cl)c(OC)c3)c(C(N)=O)c2N)C1] | 0.18 | n.d. |
| II-1 | 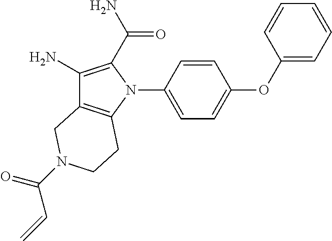 [SMILES: C=CC(=O)N1CCc2c(c(N)c(C(N)=O)n2-c2ccc(Oc3ccccc3)cc2)C1] | 3.3 | 16.6 |
| II-2 | 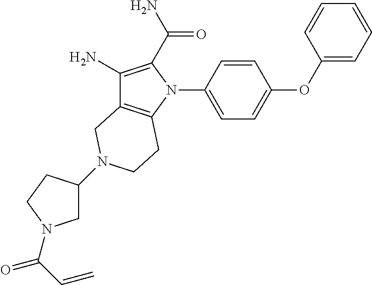 [SMILES: C=CC(=O)N1CCC(N2CCc3c(c(N)c(C(N)=O)n3-c3ccc(Oc4ccccc4)cc3)C2)C1] | 3200 | n.d. |
| II-3 | 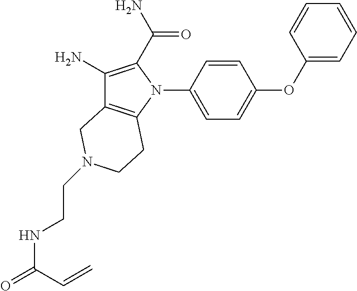 [SMILES: C=CC(=O)NCCN1CCc2c(c(N)c(C(N)=O)n2-c2ccc(Oc3ccccc3)cc2)C1] | 480 | n.d. |
| II-4 | 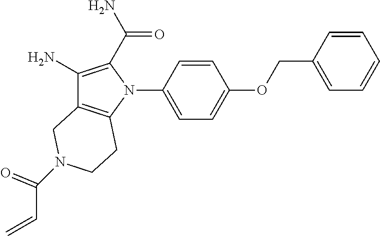 [SMILES: C=CC(=O)N1CCc2c(c(N)c(C(N)=O)n2-c2ccc(OCc3ccccc3)cc2)C1] | 25 | n.d. |
| I-5 | 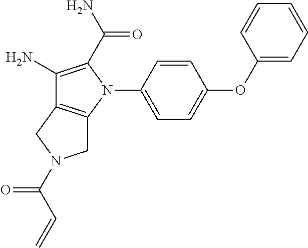 [SMILES: C=CC(=O)N1Cc2c(N)c(C(N)=O)n(-c3ccc(Oc4ccccc4)cc3)c2C1] | 9 | n.d. |
| II-6 | 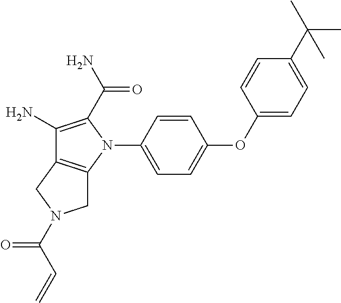 [SMILES: C=CC(=O)N1Cc2c(N)c(C(N)=O)n(-c3ccc(Oc4ccc(C(C)(C)C)cc4)cc3)c2C1] | 34 | n.d. |
| II-7 | 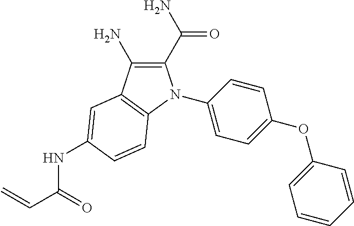 [SMILES: C=CC(=O)Nc1ccc2c(c1)c(N)c(C(N)=O)n2-c1ccc(Oc2ccccc2)cc1] | 9.4 | 55.6 |
| II-8 | 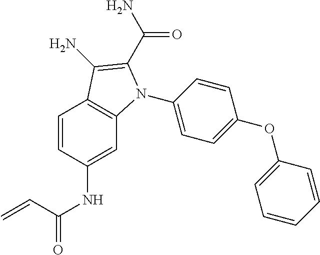 [SMILES: C=CC(=O)Nc1ccc2c(N)c(C(N)=O)n(-c3ccc(Oc4ccccc4)cc3)c2c1] | 210 | n.d. |
| II-9 | 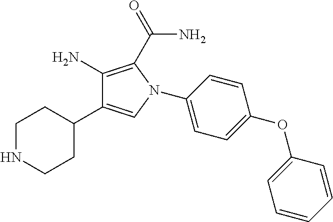 [SMILES: NC(=O)c1c(N)c(C2CCNCC2)cn1-c1ccc(Oc2ccccc2)cc1] | 7600 | n.d. |
| II-10 | 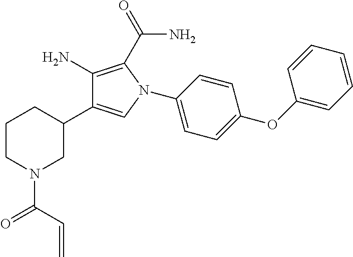 [SMILES: C=CC(=O)N1CCCC(c2cn(-c3ccc(Oc4ccccc4)cc3)c(C(N)=O)c2N)C1] | 2.7 | n.d. |

n.d. not determined

TABLE II

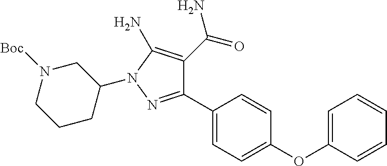 [SMILES: CC(C)(C)OC(=O)N1CCCC(n2nc(-c3ccc(Oc4ccccc4)cc3)c(C(N)=O)c2N)C1]

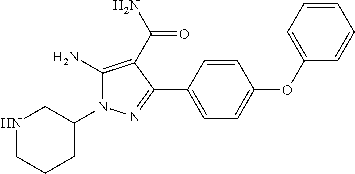 [SMILES: NC(=O)c1c(-c2ccc(Oc3ccccc3)cc2)nn(C2CCCNC2)c1N]

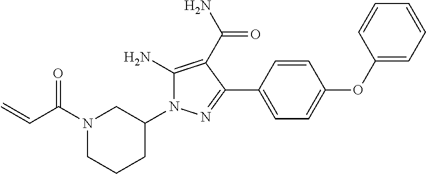 [SMILES: C=CC(=O)N1CCCC(n2nc(-c3ccc(Oc4ccccc4)cc3)c(C(N)=O)c2N)C1]

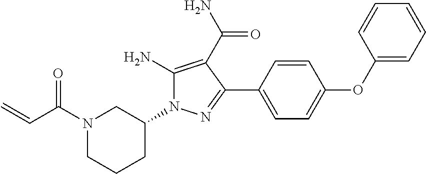 [SMILES: C=CC(=O)N1CCC[C@@H](n2nc(-c3ccc(Oc4ccccc4)cc3)c(C(N)=O)c2N)C1]

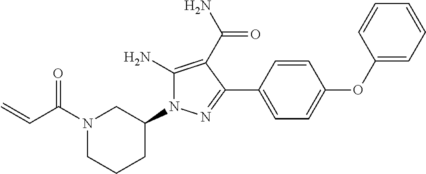 [SMILES: C=CC(=O)N1CCC[C@H](n2nc(-c3ccc(Oc4ccccc4)cc3)c(C(N)=O)c2N)C1]

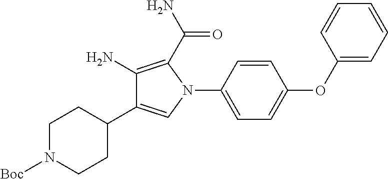 [SMILES: CC(C)(C)OC(=O)N1CCC(c2cn(-c3ccc(Oc4ccccc4)cc3)c(C(N)=O)c2N)CC1]

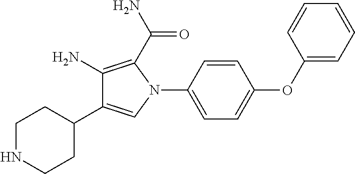 [SMILES: NC(=O)c1c(N)c(C2CCNCC2)cn1-c1ccc(Oc2ccccc2)cc1]

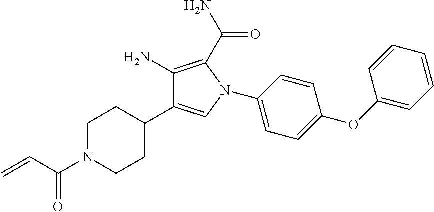 [SMILES: C=CC(=O)N1CCC(c2cn(-c3ccc(Oc4ccccc4)cc3)c(C(N)=O)c2N)CC1]

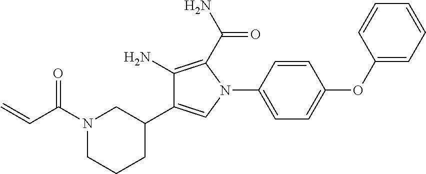 [SMILES: C=CC(=O)N1CCCC(c2cn(-c3ccc(Oc4ccccc4)cc3)c(C(N)=O)c2N)C1]

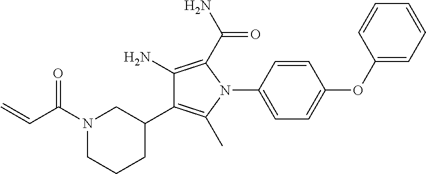 [SMILES: C=CC(=O)N1CCCC(c2c(N)c(C(N)=O)n(-c3ccc(Oc4ccccc4)cc3)c2C)C1]

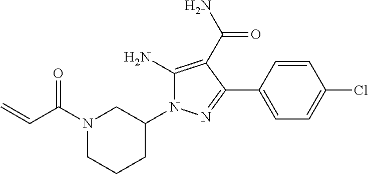 [SMILES: C=CC(=O)N1CCCC(n2nc(-c3ccc(Cl)cc3)c(C(N)=O)c2N)C1]

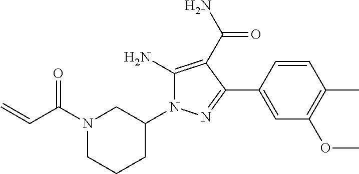 [SMILES: C=CC(=O)N1CCCC(n2nc(-c3ccc(C)c(OC)c3)c(C(N)=O)c2N)C1]

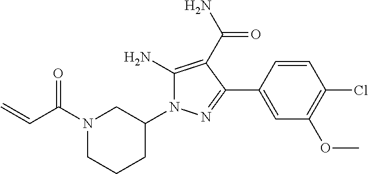 [SMILES: C=CC(=O)N1CCCC(n2nc(-c3ccc(Cl)c(OC)c3)c(C(N)=O)c2N)C1]

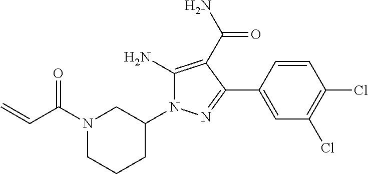 [SMILES: C=CC(=O)N1CCCC(n2nc(-c3ccc(Cl)c(Cl)c3)c(C(N)=O)c2N)C1]

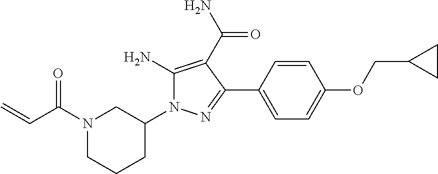 [SMILES: C=CC(=O)N1CCCC(n2nc(-c3ccc(OCC4CC4)cc3)c(C(N)=O)c2N)C1]

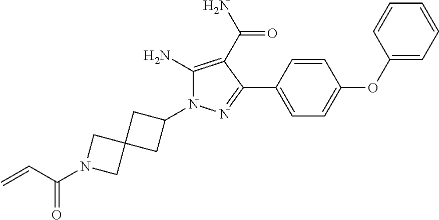 [SMILES: C=CC(=O)N1CC2(CC(n3nc(-c4ccc(Oc5ccccc5)cc4)c(C(N)=O)c3N)C2)C1]

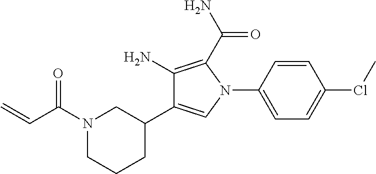

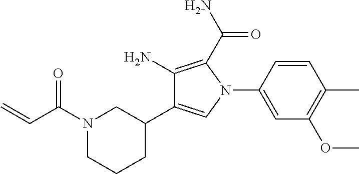 [SMILES: C=CC(=O)N1CCCC(c2cn(-c3ccc(C)c(OC)c3)c(C(N)=O)c2N)C1]

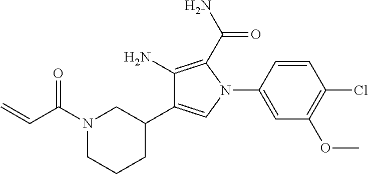 [SMILES: C=CC(=O)N1CCCC(c2cn(-c3ccc(Cl)c(OC)c3)c(C(N)=O)c2N)C1]

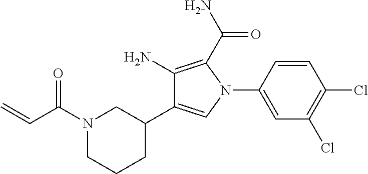 [SMILES: C=CC(=O)N1CCCC(c2cn(-c3ccc(Cl)c(Cl)c3)c(C(N)=O)c2N)C1]

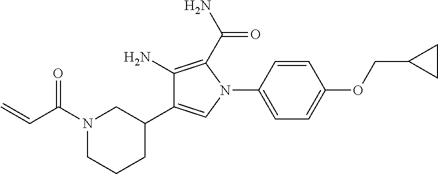 [SMILES: C=CC(=O)N1CCCC(c2cn(-c3ccc(OCC4CC4)cc3)c(C(N)=O)c2N)C1]

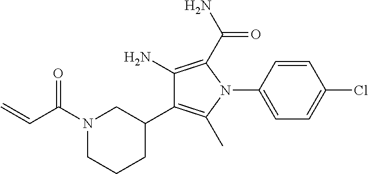 [SMILES: C=CC(=O)N1CCCC(c2c(N)c(C(N)=O)n(-c3ccc(Cl)cc3)c2C)C1]

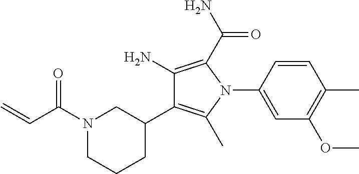 [SMILES: C=CC(=O)N1CCCC(c2c(N)c(C(N)=O)n(-c3ccc(C)c(OC)c3)c2C)C1]

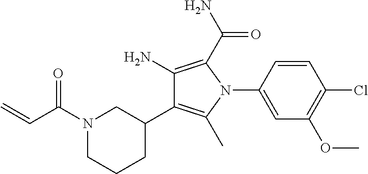 [SMILES: C=CC(=O)N1CCCC(c2c(N)c(C(N)=O)n(-c3ccc(Cl)c(OC)c3)c2C)C1]

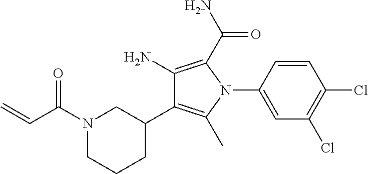 [SMILES: C=CC(=O)N1CCCC(c2c(N)c(C(N)=O)n(-c3ccc(Cl)c(Cl)c3)c2C)C1]

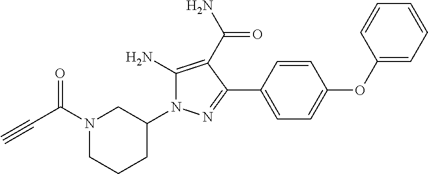 [SMILES: C#CC(=O)N1CCCC(n2nc(-c3ccc(Oc4ccccc4)cc3)c(C(N)=O)c2N)C1]

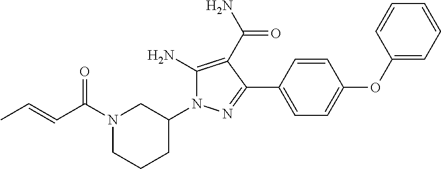 [SMILES: C/C=C/C(=O)N1CCCC(n2nc(-c3ccc(Oc4ccccc4)cc3)c(C(N)=O)c2N)C1]

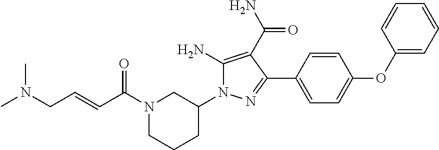 [SMILES: CN(C)C/C=C/C(=O)N1CCCC(n2nc(-c3ccc(Oc4ccccc4)cc3)c(C(N)=O)c2N)C1]

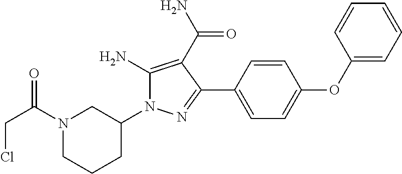 [SMILES: NC(=O)c1c(-c2ccc(Oc3ccccc3)cc2)nn(C2CCCN(C(=O)CCl)C2)c1N]

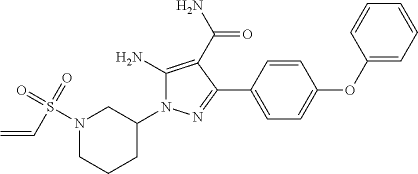 [SMILES: C=CS(=O)(=O)N1CCCC(n2nc(-c3ccc(Oc4ccccc4)cc3)c(C(N)=O)c2N)C1]

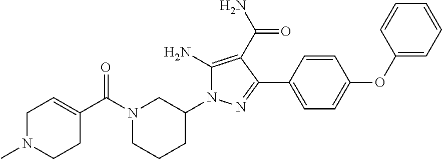 [SMILES: CN1CC=C(C(=O)N2CCCC(n3nc(-c4ccc(Oc5ccccc5)cc4)c(C(N)=O)c3N)C2)CC1]

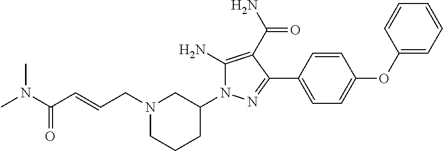 [SMILES: CN(C)C(=O)/C=C/CN1CCCC(n2nc(-c3ccc(Oc4ccccc4)cc3)c(C(N)=O)c2N)C1]

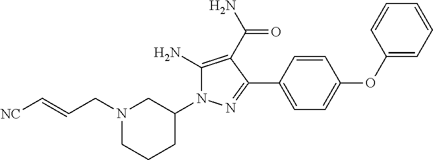 [SMILES: N#C/C=C/CN1CCCC(n2nc(-c3ccc(Oc4ccccc4)cc3)c(C(N)=O)c2N)C1]

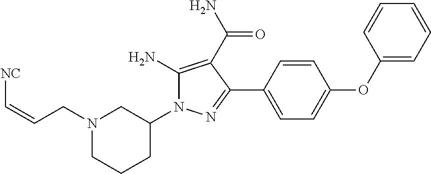

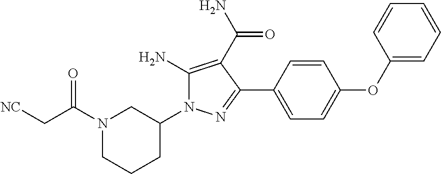 [SMILES: N#CCC(=O)N1CCCC(n2nc(-c3ccc(Oc4ccccc4)cc3)c(C(N)=O)c2N)C1]

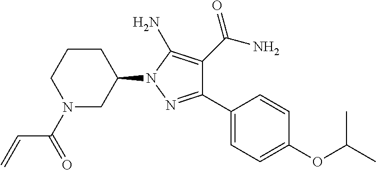 [SMILES: C=CC(=O)N1CCC[C@@H](n2nc(-c3ccc(OC(C)C)cc3)c(C(N)=O)c2N)C1]

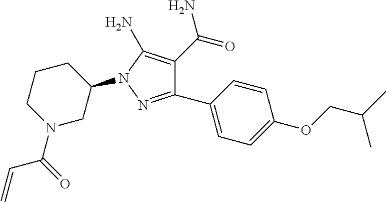 [SMILES: C=CC(=O)N1CCC[C@@H](n2nc(-c3ccc(OCC(C)C)cc3)c(C(N)=O)c2N)C1]

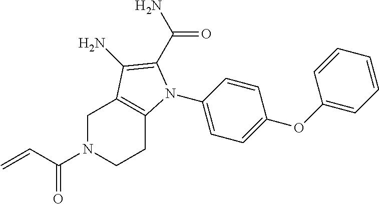 [SMILES: C=CC(=O)N1CCc2c(c(N)c(C(N)=O)n2-c2ccc(Oc3ccccc3)cc2)C1]

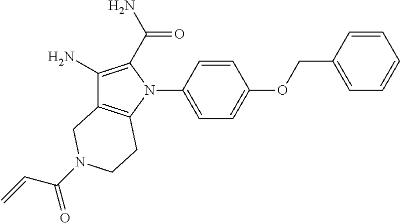 [SMILES: C=CC(=O)N1CCc2c(c(N)c(C(N)=O)n2-c2ccc(OCc3ccccc3)cc2)C1]

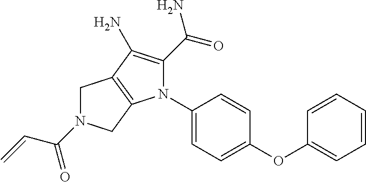 [SMILES: C=CC(=O)N1Cc2c(N)c(C(N)=O)n(-c3ccc(Oc4ccccc4)cc3)c2C1]

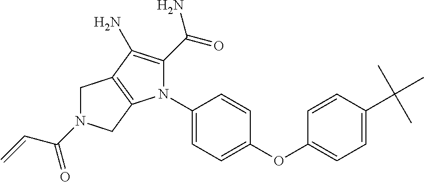 [SMILES: C=CC(=O)N1Cc2c(N)c(C(N)=O)n(-c3ccc(Oc4ccc(C(C)(C)C)cc4)cc3)c2C1]

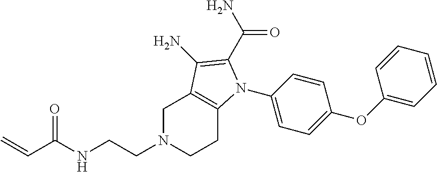 [SMILES: C=CC(=O)NCCN1CCc2c(c(N)c(C(N)=O)n2-c2ccc(Oc3ccccc3)cc2)C1]

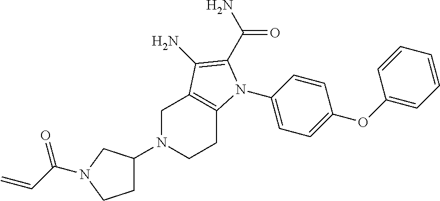 [SMILES: C=CC(=O)N1CCC(N2CCc3c(c(N)c(C(N)=O)n3-c3ccc(Oc4ccccc4)cc3)C2)C1]

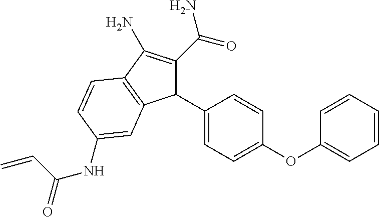 [SMILES: C=CC(=O)Nc1ccc2c(c1)C(c1ccc(Oc3ccccc3)cc1)C(C(N)=O)=C2N]

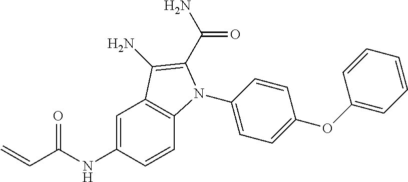 [SMILES: C=CC(=O)Nc1ccc2c(c1)c(N)c(C(N)=O)n2-c1ccc(Oc2ccccc2)cc1]

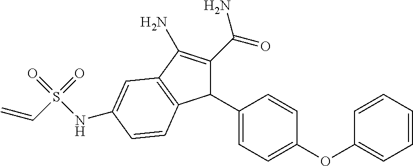 [SMILES: C=CS(=O)(=O)Nc1ccc2c(c1)C(N)=C(C(N)=O)C2c1ccc(Oc2ccccc2)cc1]

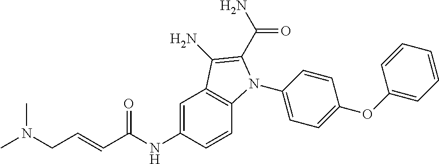 [SMILES: CN(C)C/C=C/C(=O)Nc1ccc2c(c1)c(N)c(C(N)=O)n2-c1ccc(Oc2ccccc2)cc1]

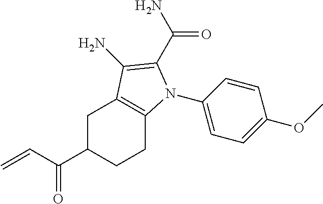 [SMILES: C=CC(=O)C1CCc2c(c(N)c(C(N)=O)n2-c2ccc(OC)cc2)C1]

What is claimed is:

1. A compound of formula: 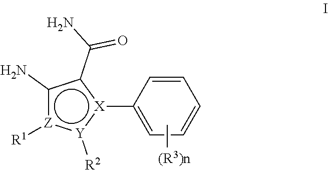
stereoisomers thereof, and pharmaceutically acceptable salts thereof, wherein:
X—Y—Z is N—C—C and R² is present, or C—N—N and R² is absent;
R¹ is 3-8 membered, N-containing ring, wherein the N is unsubstituted or substituted with R⁴;
R² is H or lower alkyl; or
R¹ and R², together with the atoms to which they are attached, form a 4-8 membered ring selected from cycloalkyl, saturated or unsaturated heterocycle, aryl, and heteroaryl rings unsubstituted or substituted with at least one substituent L-R⁴;
R³ is in each instance, independently halogen, alkyl, S-alkyl, CN, OR⁵;
n is 1, 2, 3 or 4;
L is a bond, NH, heteroalkyl, or heterocyclyl;
R⁴ is COR′, CO₂R′, or SO₂R′, wherein R′ is substituted or unsubstituted alkyl, substituted or unsubstituted alkenyl, substituted or unsubstituted alkynyl; and
R⁵ is H, or unsubstituted or substituted heteroalkyl, alkyl, cycloalkyl, saturated or unsaturated heterocyclyl, aryl, or heteroaryl.

2. The compound of claim 1, wherein:
X—Y—Z is C—N—N and R² is absent; and
R¹ is 3-8 membered, N-containing ring, N-substituted with R⁴.

3. The compound of claim 1, wherein:
X—Y—Z is N—C—C and R² is present,
R¹ is 3-8 membered, N-containing ring, N-substituted with R⁴; and
R² is H or lower alkyl.

4. The compound of claim 1, wherein:
X—Y—Z is N—C—C and R² is present; and
R¹ and R², together with the atoms to which they are attached, form a 4-8 membered ring selected from cycloalkyl, saturated or unsaturated heterocycle, aryl, and heteroaryl rings unsubstituted or substituted with at least one substituent L-R⁴.

5. The compound of claim 1, wherein:
X—Y—Z is N—C—C and R² is present; and
R¹ and R², together with the atoms to which they are attached, form a 4-8 membered ring that is (a) phenyl substituted with a single -L-R⁴, or (b) dihydropyrrole or tetrahydropyridine, N-substituted with a single -L-R⁴ wherein L is bond.

6. The compound of claim 1 wherein:
R¹ is piperidine or azaspiro[3.3]heptane, N-substituted with R⁴.

7. The compound of claim 1, wherein:
R¹ is piperidine or azaspiro[3.3]heptane, N-substituted with R⁴; and
R⁴ is COR′ or SO₂R′, and R′ is substituted or unsubstituted alkenyl.

8. The compound of claim 1, wherein:
R³ is —OR⁵, R⁵ is phenyl, and n is 1.

9. The compound of claim 1, wherein:
X—Y—Z is C—N—N and R² is absent;
R¹ is piperidine, N-substituted with R⁴;
R³ is —OR⁵;
n is 1;
R⁴ is COR′, and R′ is ethenyl; and
R⁵ is phenyl.

10. A compound of the examples herein, or of Table I or II, stereoisomers thereof, and pharmaceutically acceptable salts thereof.

11. A compound below, or a pharmaceutically acceptable salt thereof:

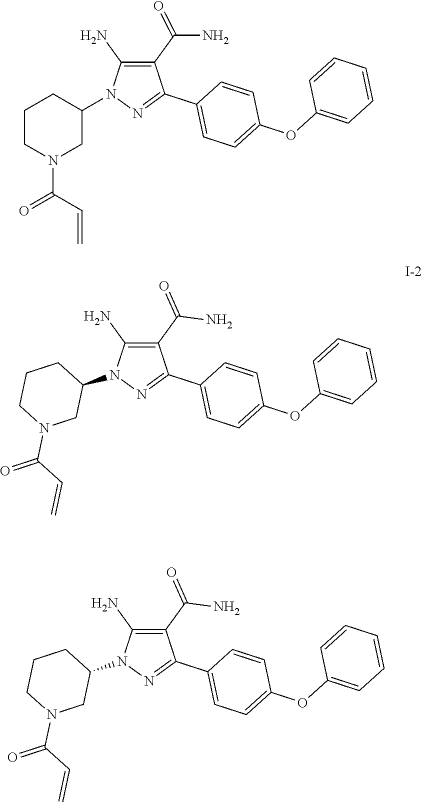 [SMILES: C=CC(=O)N1CCCC(n2nc(-c3ccc(Oc4ccccc4)cc3)c(C(N)=O)c2N)C1 ; C=CC(=O)N1CCC[C@@H](n2nc(-c3ccc(Oc4ccccc4)cc3)c(C(N)=O)c2N)C1 ; C=CC(=O)N1CCC[C@H](n2nc(-c3ccc(Oc4ccccc4)cc3)c(C(N)=O)c2N)C1]

12. The compound of claim 1, having a Btk-inhibiting activity corresponding to an IC50 of 10 uM or less in a Btk Kinase Assay.

13. A pharmaceutical composition comprising a therapeutically effective amount of a compound of claim 1 in unit dosage form and one or more pharmaceutically acceptable carriers.

14. A combination comprising a therapeutically effective amount of a compound of claim 1 and a different agent therapeutically active against an autoimmune and/or inflammatory disease or cancer.

15. A method of treating a disease associated with undesirable Btk activity, which comprises administering to a person in need thereof an effective amount of a compound of claim 1, an N-oxide thereof or a prodrug thereof.
